(19) United States
(12) Patent Application Publication
(10) Pub. No.: US 2026/0036401 A1
(43) Pub. Date: Feb. 5, 2026

(54) REAL-TIME EVIDENCE MANAGEMENT SYSTEM FOR CODE ENFORCEMENT USING BODY-WORN AND VEHICLE-MOUNTED AI CAMERAS

(71) Applicant: GovernmentGPT, INC, Mountain View, CA (US)

(72) Inventor: Raj Abhyanker, Mountain View, CA (US)

(21) Appl. No.: 19/354,873

(22) Filed: Oct. 10, 2025

Related U.S. Application Data
(63) Continuation-in-part of application No. 19/235,611, filed on Jun. 12, 2025.
(63) Continuation of application No. 18/596,684, filed on Mar. 6, 2024, now Pat. No. 12,392,583.
(60) Provisional application No. 63/614,022, filed on Dec. 22, 2023.
(60) Provisional application No. 63/616,817, filed on Jan. 1, 2024.
(60) Provisional application No. 63/622,514, filed on Jan. 18, 2024.
(60) Provisional application No. 63/626,075, filed on Jan. 29, 2024.
(60) Provisional application No. 63/552,265, filed on Feb. 12, 2024.
(60) Provisional application No. 63/554,360, filed on Feb. 16, 2024.
(60) Provisional application No. 63/555,014, filed on Feb. 17, 2024.

(51) Int. Cl. F41H 1/00; A61B 5/00; G06F 40/58; G08B 6/00; G08B 21/02

(52) U.S. Cl. CPC F41H 1/00; A61B 5/6805; A61B 5/747; G06F 40/58; G08B 6/00; G08B 21/02

(57) ABSTRACT

A method, apparatus, and system of automated code enforcement monitoring using geospatially tagged video capture is disclosed. In one embodiment, a data acquisition device is provided comprising at least one of a body-worn camera worn by a code enforcement officer, a vehicle-mounted camera, or a drone deployed from the vehicle. The device captures video data of real property together with geospatial coordinates and timestamps within a jurisdictional boundary. An evidence management server is communicatively coupled to the device through a network to store the video data, coordinates, and timestamps. The server identifies a parcel number associated with the captured property based on geospatial coordinates. A violation detection module compares timestamped video data of the parcel with previously captured data to determine modifications to a physical structure or landscaping, thereby identifying potential violations of jurisdictional codes.

### CLAIM OF PRIORITY

This Application is a Continuation-In-Part Application of, and claims priority to, co-pending U.S. patent application Ser. No. 19/235,611 titled BODY-WORN CAMERA SYSTEM WITH INTEGRATED ARTIFICIAL INTELLIGENCE FOR REALTIME FIELD ASSISTANCE AND AUTOMATED INCIDENT REPORTING filed on Jun. 12, 2025, which is a Continuation Application of, and claims priority to, the granted U.S. patent Ser. No. 12/392,583 titled BODY SAFETY DEVICE WITH VISUAL SENSING AND HAPTIC RESPONSE USING ARTIFICIAL INTELLIGENCE filed on Mar. 6, 2024, which is a conversion Application of and claims priority to, and incorporated by reference herein the entirety of the disclosures of:
U.S. Provisional Patent Application No. 63/614,022 titled MULTI-FUNCTIONAL WEARABLE AI-ENABLED PENDANT APPARATUS, SYSTEM, AND METHOD OF AMBIENT DATA ANALYSIS AND COMMUNICATION IN LAW ENFORCEMENT, FIRE, MEDICAL RESPONDER, PRIVATE SECURITY, JOURNALISM, COMMERCIAL AND MILITARY OPERATIONAL ENVIRONMENTS filed on Dec. 22, 2023 whose entirety of disclosure was incorporated fully into reference in the parent application Ser. No. 18/596,684 filed on Mar. 6, 2024, now granted U.S. patent Ser. No. 12/392,583,
U.S. Provisional Patent Application No. 63/616,817 titled EMOTIONALLY INTELLIGENT AERIAL DRONE SYSTEM FOR ENHANCED SITUATIONAL AWARENESS AND RESPONSIVE OPERATIONS filed on Jan. 1, 2024,
U.S. Provisional Patent Application No. 63/622,514 titled HAPTIC FEEDBACK RESPONSIVE TO A THREAT IDENTIFIED THROUGH A GENERATIVE ARTIFICIAL INTELLIGENCE BODY WORN APPARATUS filed on Jan. 18, 2024,
U.S. Provisional Patent Application No. 63/626,075 titled SECURE EDGE MESH NETWORK SYSTEM FOR ENHANCED VISUAL INTERPRETATION AND REAL-TIME SITUATIONAL AWARENESS IN COMBAT ZONES filed on Jan. 29, 2024,
U.S. Provisional Patent Application No. 63/552,265 titled MODULAR INTEGRATED BODY CAMERA SYSTEM FOR ENHANCED ERGONOMICS, OPERATIONAL EFFICIENCY, AND TECHNOLOGICAL ADAPTABILITY IN LAW ENFORCEMENT EQUIPMENT filed on Feb. 12, 2024,
U.S. Provisional Patent Application No. 63/554,360 titled ENHANCED SITUATIONAL AWARENESS THROUGH A HAPTIC WEARABLE DEVICE OF A POLICE OFFICER OR A WARFIGHTER, ACTIVATED BY A NEARBY NETWORKED VEHICLE OR A STATIONARY SENSOR UPON DETECTING A THREAT filed on Feb. 16, 2024; and
U.S. Provisional Patent Application No. 63/555,014 titled TRAUMATIC INJURY COMMUNICATION METHODOLOGY AND SYSTEM THROUGH A BODY WORN DEVICE filed on Feb. 17, 2024.

### FIELD OF TECHNOLOGY

The present disclosure generally relates to the field of real estate compliance and regulatory enforcement, more particularly, to a system, method, and apparatus to detect changes in physical property characteristics and identify potential code violations using artificial intelligence (AI), computer vision, and data collected from body-worn and vehicle-mounted cameras operated by code enforcement personnel.

### BACKGROUND

When municipalities are resource-constrained and lack sufficient inspectors, a range of serious problems can emerge that affect safety, housing quality, and/or public trust. Uninspected construction projects may result in buildings that do not meet fire, electrical, and/or structural codes, which can increase the likelihood of accidents, injuries, and/or fatalities. Unsafe wiring, inadequate fire exits, and/or poor structural integrity can go undetected, creating risks for both residents and emergency personnel.

In areas where enforcement is limited, landlords may convert garages and/or sheds into illegal dwellings without oversight. These unpermitted units can lack proper ventilation, plumbing, and/or fire protection, exposing tenants to unsafe living conditions. Low-income and/or immigrant populations may be disproportionately affected, as they often have fewer housing options and may hesitate to report violations due to fear of retaliation and/or eviction.

Limited inspection capacity can also lead to unequal enforcement. Code enforcement may become reactive, responding primarily to complaints. This dynamic can result in violations being addressed only in neighborhoods with more political influence and/or public pressure, while other communities are overlooked. Over time, this may erode trust in government institutions and create a perception of bias and/or neglect.

Developers and/or property owners who proceed without permits can gain an unfair economic advantage. This may discourage compliance by others and weaken the credibility of municipal regulation. Legal builders who follow the rules may be undercut by those who bypass oversight, damaging the integrity of the local construction industry.

Municipalities may also lose substantial revenue due to uncollected permit fees, inspection charges, and fines. At the same time, they can face increased liability exposure if accidents and/or structural failures occur in properties that should have been inspected. Insurance costs may rise for both public agencies and private owners as a result of unaddressed hazards.

Unauthorized development can strain infrastructure, such as sewers, roads, and/or emergency services. When population growth occurs through illegal and/or unplanned construction, cities may find themselves unprepared to meet demand for basic services. This can reduce quality of life and lead to long-term maintenance issues.

Finally, visible code violations that persist over time can contribute to urban decay. Neglected properties may deter investment, reduce nearby property values, and create environments where crime and disrepair flourish. Without adequate enforcement, the built environment may degrade, affecting not only individual neighborhoods but the health and reputation of the municipality as a whole.

### SUMMARY

Disclosed are a method, a device and/or a system of a real-time evidence management system for code enforcement using body-worn and vehicle-mounted AI cameras.

In one aspect, a system includes a data acquisition device that includes a body-worn camera wearable by a code enforcement officer, a vehicle-mounted camera in a vehicle of the code enforcement officer, and/or a vehicle-deployed drone from the vehicle, each configured to capture video data from a real property along with geospatial coordinates of where the video data is captured in a jurisdictional boundary and a timestamp of when the video data is captured. The system further includes an evidence management server communicatively coupled with the data acquisition device through a network to: (1) store the video data captured by the data acquisition device along with the geospatial coordinates and the timestamp, (2) identify a parcel number in the jurisdictional boundary associated with a parcel captured in the video data based on the geospatial coordinates, (3) determine whether there is a violation of a jurisdictional code based on a modification of a physical structure and/or a landscaping in the parcel when the timestamp is compared to the immediately previous timestamp of video footage captured associated with the same parcel number.

The system may include the body-worn camera and/or the vehicle-deployed drone launchable from the vehicle. The vehicle-deployed drone may be configured to capture an aerial image and/or the video data of the real property and to transmit the aerial image and/or the video data with the geospatial coordinates to the evidence management server, optionally in real time. The evidence management server may include an artificial intelligence module configured to analyze the aerial image and/or the video data to detect a violation, and the artificial intelligence module may be configured to generate a real-time alert to the code enforcement officer upon detecting the violation. The body-worn camera and/or the vehicle-mounted camera may be configured to operate in a synchronized mode to capture multiple perspectives of the real property simultaneously and to provide time-aligned frames to the evidence management server. The evidence management server may include an evidence-management interface configured to integrate with a municipal database to automatically cross-reference a detected change with a property record and/or a permit record associated with the parcel number. The evidence management server may further include a geolocation module configured to associate captured images and/or the video data with a geographic location of the real property and to map the detected change to a parcel-boundary polygon corresponding to the parcel number. The evidence management server may include a computer-vision module configured to detect at least one of construction of an unpermitted structure, alteration to an exterior building characteristic, accumulation of debris, non-compliant materials, and/or modification to fencing, driveways, and/or accessory structures. The evidence-management interface may further include an automated report generator configured to prepare an enforcement notice and citation based on an identified violation, the enforcement notice and citation may include a time-stamped, geo-referenced image frame captured by at least one of the body-worn camera, the vehicle-deployed drone, and/or an ordinance citation. The evidence management server may be configured to detect interior modifications of the real property based on imagery obtained through windows, open structures, and/or authorized interior inspections, and to associate each detection with a confidence score.

In another aspect, a system includes a body-worn camera wearable by a code enforcement officer that is configured to capture video data of a real property together with a geospatial coordinate and a timestamp. The system includes a vehicle-deployed drone launchable from a vehicle that is configured to capture an aerial image and/or the aerial video data of the real property together with the geospatial coordinates and the timestamp. The system includes an evidence management server communicatively coupled with the body-worn camera and/or the vehicle-deployed drone through a network. The evidence management server is configured to: (1) receive the video data from the body-worn camera and/or the aerial video data from the vehicle-deployed drone. (2) Time-synchronize the video data and/or the aerial video data. (3) Identify a parcel number associated with the real property based on the geospatial coordinates. (4) Compare a time-synchronized data with the video data associated with an immediately previous timestamp for the parcel number to detect a modification to at least one of a physical structure and/or a landscaping. (5) Evaluate a detected modification against digitized municipal code sections and cross-reference the detected modification with the real property and/or permit records retrieved from encrypted copies and/or a municipal database. (6) Generate a real-time alert responsive to a determination of non-compliance. (7) Create an enforcement notice including geo-referenced imagery captured by at least one of the body-worn camera and/or the vehicle-deployed drone.

The system may include an evidence management server that may comprise a municipal dashboard configured to present active violations, owner information, prior permits, prior violations for the parcel number, and/or a priority ranking based on a severity level, a location risk factor, and/or a duration since detection. The evidence management server may further comprise a change-detection module configured to perform semantic segmentation, pixel-wise differencing, and/or bounding-box generation to isolate a detected portion and to compute a measurement selected from width, height, surface area, and/or footprint boundary, and to overlay the measurement on the image and/or the video data. The vehicle-deployed drone may comprise a gimbal-stabilized camera and a telemetry transceiver and may be configured to stream live geo-tagged video to the evidence management server. The evidence management server may be further configured to command the vehicle-deployed drone to reposition to capture a supplemental aerial view of the detected portion and to fuse the supplemental aerial view with a time-synchronized video from the body-worn camera. The evidence management server may be configured to transmit an automated notification of a violation via electronic mail, short message service, a municipal web portal, and/or printed mail, and to schedule a site visit by a municipal staff member responsive to the real-time alerts. The evidence management server may be configured to assemble an evidence package including the enforcement notice, before-and-after imagery with overlaid measurements, the geospatial coordinates, an applicable ordinance citation, and chain-of-custody metadata including a cryptographic hash and/or a tamper-evident audit log. The evidence management server may comprise a machine-learning model trained on historical enforcement outcomes and property imagery and may be configured to update detection performance by periodic retraining using labeled compliant and/or non-compliant events. The body-worn camera and/or the vehicle-deployed drone may be time-synchronized using a common clock distributed by the evidence management server to enable frame-level correlation and multi-perspective fusion.

In yet another aspect, a computer-implemented method for real-time evidence management, the method includes, (1) storing in a jurisdictional code database a jurisdictional regulation in an evidence management server comprising any one or more of a planning code, a building code, a construction code, a public works code, a cleanliness code, an administrative code, a statutory code, a fire code, a police code, an ordinance, a safety code, a housing code, a municipal code. (2) capturing a video data of a real property using at least one of a body-worn camera worn by a code enforcement officer, a vehicle-mounted camera in a vehicle of the code enforcement officer, and a vehicle-deployed drone launched from the vehicle, the video data including a geospatial coordinate within a jurisdictional boundary and a timestamp of capture. (3) transmitting the video data, the geospatial coordinates, and a timestamp from a data acquisition device to the evidence management server through a network. (4) storing the video data together with the geospatial coordinates and the timestamp in the evidence management server. (5) identifying a parcel number in the jurisdictional boundary that corresponds to the real property captured in the video data based on the geospatial coordinates. (6) determining whether a violation of the jurisdictional regulation has occurred by comparing a timestamped video data with the video data from an immediately previous timestamp associated with the same parcel number to detect a modification to at least one of a physical structure and a landscaping of a parcel.

The method may include a multispectral imaging module configured to capture at least one of infrared imagery, thermal imagery, and/or LiDAR point clouds. The evidence management server may be configured to detect structural changes not visible in RGB imagery, including at least one of roof degradation, heat leakage, and/or energy code violations. The method may further include an acoustic sensing module configured to detect non-compliant sound patterns, the non-compliant sound patterns comprising at least one of construction machinery operated outside permitted hours, amplified sound levels exceeding municipal noise ordinances, and/or unpermitted industrial equipment.

The evidence management server may be communicatively coupled to at least one environmental sensor configured to detect air quality, dust generation, light emissions, and/or waste disposal conditions, and to evaluate compliance with an environmental regulation associated with the parcel number, and may be configured to fuse the video data from the data acquisition device with resident-reported imagery comprising photographs, video, and/or sensor recordings submitted through a municipal reporting portal to corroborate detection of a jurisdictional code violation. An artificial intelligence module may comprise a natural language processing engine configured to automatically map a detected modification to a specific section of a digitized municipal ordinance and/or regulatory code.

The artificial intelligence module may further comprise a consensus engine configured to execute a plurality of trained detection models and to issue a violation alert only upon multi-model consensus exceeding a confidence threshold. The evidence management server may be configured to generate a three-dimensional parcel model by aggregating repeated aerial captures from the vehicle-deployed drone and to detect volumetric changes to the parcel, including footprint expansion, height increases, and/or demolition. The evidence management server may further comprise a heat-mapping module configured to visualize detected violations on a jurisdictional map, assigning severity levels and/or risk indicators to prioritize enforcement actions, and a machine-learning model that may retrain adaptively using enforcement outcomes, including citations upheld and/or dismissed, to improve future detection accuracy.

The evidence management server may overlay detected parcel modifications against zoning maps to identify mismatches between actual property usage and designated zoning classifications. An evidence package may comprise a multi-tier encrypted copy of sensitive resident-facing imagery, the multi-tier encryption comprising a redacted public copy and an unredacted secured chain-of-custody copy, and may further comprise a digitally watermarked image frame embedding the parcel number, ordinance citation, timestamp, and/or geospatial coordinates directly into an evidentiary image.

The evidence management server may also be configured to provide an augmented reality overlay to a display associated with the body-worn camera, visually superimposing detected violations and/or ordinance references onto a real-world scene in real time. The data acquisition device may further comprise a voice annotation module enabling the code enforcement officer to dictate contextual notes while recording, the notes being transcribed, geotagged, and automatically linked to the corresponding evidentiary record.

### BRIEF DESCRIPTION OF FIGURES

The embodiments of this invention are illustrated by way of example and not limitation in the figures of the accompanying drawings, in which like references indicate similar elements and in which:

FIG. 1 is a system architecture view of the automated code enforcement system illustrating the data acquisition devices, evidence management server, municipal dashboard interface, and automated processing modules used to detect, record, and manage municipal code violations, according to one embodiment.

FIG. 2 is a sensor integration and data flow view illustrating how acoustic sensing, environmental monitoring, and telemetry devices communicate over a network with the evidence management server to detect changes and synchronize timestamps for accurate code enforcement analysis, according to one embodiment.

FIG. 3 is a functional process view illustrating code violation detection and evidence assembly, showing how digitized code databases, permit records, and parcel data interact with the evidence management interface to identify and document violations, according to one embodiment.

FIG. 4 is a workflow view of the enforcement notice and evidence packaging process, illustrating the generation of a zoning violation notice, linkage to geographic coordinates, and the assembly of supporting data such as audit logs, encrypted copies, and watermarked images, according to one embodiment.

FIG. 5 is a video data synchronization view illustrating the capture and alignment of supplemental aerial views, live geo-tagged video, and ground-level footage using drones and server-based processing, according to one embodiment.

FIG. 6 is a volumetric analysis workflow view illustrating the use of repeated aerial captures to build three-dimensional parcel models, detect volumetric changes, and compute metrics such as height, footprint, and volume differences for code compliance purposes, according to one embodiment.

FIG. 7 is a jurisdictional heat mapping and prioritization view illustrating risk-based categorization of parcels based on severity and urgency of violations, with graphical overlays and textual rankings to guide enforcement strategy, according to one embodiment.

FIG. 8 is a visual scene analysis and AI-based detection overlay view illustrating how artificial intelligence and computer-vision modules identify specific violations such as unpermitted structures, vegetation overgrowth, and trash accumulation, together with location metadata and confidence scores, according to one embodiment.

Other features of the present embodiments will be apparent from the accompanying drawings and from the detailed description that follows.

### DETAILED DESCRIPTION

In one embodiment, a system includes a data acquisition device 102 that includes a body-worn camera 106 wearable by a code enforcement officer 104, a vehicle-mounted camera 112 in a vehicle 110 of the code enforcement officer 104, and/or a vehicle-deployed drone 114 from the vehicle 110, each configured to capture video data from a real property 120 along with geospatial coordinates 126 of where the video data is captured in a jurisdictional boundary and a timestamp 128 of when the video data is captured. The system further includes an evidence management server 132 communicatively coupled with the data acquisition device 102 through a network 130 to: (1) store the video data captured by the data acquisition device 102 along with the geospatial coordinates 126 and the timestamp 128, (2) identify a parcel number 124 in the jurisdictional boundary associated with a parcel captured in the video data based on the geospatial coordinates 126, (3) determine whether there is a violation of a jurisdictional code based on a modification of a physical structure and/or a landscaping in the parcel when the timestamp 128 is compared to the immediately previous timestamp 128 of video footage captured associated with the same parcel number 124.

The system may include the body-worn camera 106 and/or the vehicle-deployed drone 114 launchable from the vehicle 110. The vehicle-deployed drone 114 may be configured to capture an aerial image and/or the video data of the real property 120 and to transmit the aerial image and/or the video data with the geospatial coordinates 126 to the evidence management server 132, optionally in real time. The evidence management server 132 may include an artificial intelligence module 134 configured to analyze the aerial image and/or the video data to detect a violation, and the artificial intelligence module 134 may be configured to generate a real-time alert to the code enforcement officer 104 upon detecting the violation. The body-worn camera 106 and/or the vehicle-mounted camera 112 may be configured to operate in a synchronized mode to capture multiple perspectives of the real property 120 simultaneously and to provide time-aligned frames to the evidence management server 132. The evidence management server 132 may include an evidence-management interface 146 configured to integrate with a municipal database 312 to automatically cross-reference a detected change with a property record 314 and/or a permit record 316 associated with the parcel number 124. The evidence management server 132 may further include a geolocation module 144 configured to associate captured images and/or the video data with a geographic location of the real property 120 and to map the detected change to a parcel-boundary polygon 122 corresponding to the parcel number 124. The evidence management server 132 may include a computer-vision module 136 configured to detect at least one of construction of an unpermitted structure, alteration to an exterior building characteristic, accumulation of debris, non-compliant materials, and/or modification to fencing, driveways, and/or accessory structures. The evidence-management interface 146 may further include an automated report generator 310 configured to prepare an enforcement notice 402 and citation 166 based on an identified violation, the enforcement notice 402 and citation 166 may include a time-stamped, geo-referenced image frame captured by at least one of the body-worn camera 106, the vehicle-deployed drone 114, and/or an ordinance citation 170. The evidence management server 132 may be configured to detect interior modifications of the real property 120 based on imagery obtained through windows, open structures, and/or authorized interior inspections, and to associate each detection with a confidence score.

In another embodiment, a system includes a body-worn camera 106 wearable by a code enforcement officer 104 that is configured to capture video data of a real property 120 together with a geospatial coordinate 126 and a timestamp 128. The system includes a vehicle-deployed drone 114 launchable from a vehicle 110 that is configured to capture an aerial image and/or the aerial video data of the real property 120 together with the geospatial coordinates 126 and the timestamp 128. The system includes an evidence management server 132 communicatively coupled with the body-worn camera 106 and/or the vehicle-deployed drone 114 through a network 130. The evidence management server 132 is configured to: (1) receive the video data from the body-worn camera 106 and/or the aerial video data from the vehicle-deployed drone 114, (2) time-synchronize the video data and/or the aerial video data using a common clock 212, (3) identify a parcel number 124 associated with the real property 120 based on the geospatial coordinates 126, (4) compare a time-synchronized data with the video data associated with an immediately previous timestamp 128 for the parcel number 124 to detect a modification to at least one of a physical structure and/or a landscaping, (5) evaluate a detected modification against digitized municipal code sections in a jurisdictional code database 152 and cross-reference the detected modification with the real property 120 and/or permit records 316 retrieved from encrypted copies 174 and/or a municipal database 312, (6) generate a real-time alert responsive to a determination of non-compliance, (7) create an enforcement notice 402 including geo-referenced imagery captured by at least one of the body-worn camera 106 and/or the vehicle-deployed drone 114.

The system may include an evidence management server 132 that may comprise a municipal dashboard 148 configured to present active violations, owner information, prior permits, prior violations for the parcel number 124, and/or a priority ranking 708 based on a severity level, a location risk factor, and/or a duration since detection. The evidence management server 132 may further comprise a change-detection module 142 configured to perform semantic segmentation, pixel-wise differencing, and/or bounding-box generation to isolate a detected portion 218 and to compute a measurement selected from width, height, surface area, and/or footprint boundary, and to overlay the measurement on the image and/or the video data. The vehicle-deployed drone 114 may comprise a gimbal-stabilized camera 118 and a telemetry transceiver 116 and may be configured to stream live geo-tagged video 504 to the evidence management server 132. The evidence management server 132 may be further configured to command the vehicle-deployed drone 114 to reposition to capture a supplemental aerial view 502 of the detected portion 218 and to fuse the supplemental aerial view 502 with a time-synchronized video from the body-worn camera 106 using a fusion module 508. The evidence management server 132 may be configured to transmit an automated notification 510 of a violation via electronic mail, short message service, a municipal web portal, and/or printed mail, and to schedule a site visit 178 by a municipal staff member responsive to the real-time alerts. The evidence management server 132 may be configured to assemble an evidence package 164 including the enforcement notice 402, before-and-after imagery with overlaid measurements 168, the geospatial coordinates 126, an applicable ordinance citation 170, and chain-of-custody metadata 172 including a cryptographic hash and/or a tamper-evident audit log 404. The evidence management server 132 may comprise a machine-learning model 154 trained on historical enforcement outcomes and property imagery and may be configured to update detection performance by periodic retraining using labeled compliant and/or non-compliant events. The body-worn camera 106 and/or the vehicle-deployed drone 114 may be time-synchronized using the common clock 212 distributed by the evidence management server 132 to enable frame-level correlation and multi-perspective fusion.

In yet another embodiment, a computer-implemented method for real-time evidence management, the method includes, (1) storing in a jurisdictional code database 152 a jurisdictional regulation in an evidence management server 132 comprising any one or more of a planning code, a building code 304, a construction code, a public works code, a cleanliness code 308, an administrative code, a statutory code, a fire code, a police code, an ordinance, a safety code, a housing code, a municipal code, (2) capturing a video data of a real property 120 using at least one of a body-worn camera 106 worn by a code enforcement officer 104, a vehicle-mounted camera 112 in a vehicle 110 of the code enforcement officer 104, and a vehicle-deployed drone 114 launched from the vehicle 110, the video data including a geospatial coordinate 126 within a jurisdictional boundary and a timestamp 128 of capture, (3) transmitting the video data, the geospatial coordinates 126, and a timestamp 128 from a data acquisition device 102 to the evidence management server 132 through a network 130, (4) storing the video data together with the geospatial coordinates 126 and the timestamp 128 in the evidence management server 132, (5) identifying a parcel number 124 in the jurisdictional boundary that corresponds to the real property 120 captured in the video data based on the geospatial coordinates 126, (6) determining whether a violation of the jurisdictional regulation has occurred by comparing a timestamped video data with the video data from an immediately previous timestamp 128 associated with the same parcel number 124 to detect a modification to at least one of a physical structure and a landscaping of a parcel.

The method may include a multispectral imaging module configured to capture at least one of infrared imagery, thermal imagery, and/or LiDAR point clouds. The evidence management server 132 may be configured to detect structural changes not visible in RGB imagery, including at least one of roof degradation, heat leakage, and/or energy code violations. The method may further include an acoustic sensing module 202 configured to detect non-compliant sound patterns, the non-compliant sound patterns comprising at least one of construction machinery operated outside permitted hours, amplified sound levels exceeding municipal noise ordinances, and/or unpermitted industrial equipment.

The evidence management server 132 may be communicatively coupled to at least one environmental sensor 204 configured to detect air quality, dust generation, light emissions, and/or waste disposal conditions, and to evaluate compliance with an environmental regulation associated with the parcel number 124, and may be configured to fuse the video data from the data acquisition device 102 with resident-reported imagery 208 comprising photographs, video, and/or sensor recordings submitted through a municipal reporting portal 206 to corroborate detection of a jurisdictional code violation. An artificial intelligence module 134 may comprise a natural language processing engine 138 configured to automatically map a detected modification to a specific section of a digitized municipal ordinance and/or regulatory code.

The artificial intelligence module 134 may further comprise a consensus engine 140 configured to execute a plurality of trained detection models and to issue a violation alert only upon multi-model consensus exceeding a confidence threshold. The evidence management server 132 may be configured to generate a three-dimensional parcel model 604 by aggregating repeated aerial captures 602 from the vehicle-deployed drone 114 and to detect volumetric changes 606 to the parcel, including footprint expansion, height increases, and/or demolition. The evidence management server 132 may further comprise a heat-mapping module 156 configured to visualize detected violations on a jurisdictional map 704, assigning severity levels and/or risk indicators 706 to prioritize enforcement actions, and a machine-learning model 154 that may retrain adaptively using enforcement outcomes, including citations upheld and/or dismissed, to improve future detection accuracy.

The evidence management server 132 may overlay detected parcel modifications against zoning maps 306 to identify mismatches between actual property usage and designated zoning classifications. An evidence package 164 may comprise a multi-tier encrypted copy 174 of sensitive resident-facing imagery, the multi-tier encryption comprising a redacted public copy and an unredacted secured chain-of-custody copy, and may further comprise a digitally watermarked image frame 176 embedding the parcel number 124, ordinance citation 170, timestamp 128, and/or geospatial coordinates 126 directly into an evidentiary image.

The evidence management server 132 may also be configured to provide an augmented reality overlay 158 to a display associated with the body-worn camera 106, visually superimposing detected violations and/or ordinance references onto a real-world scene in real time. The data acquisition device 102 may further comprise a voice annotation module 108 enabling the code enforcement officer 104 to dictate contextual notes while recording, the notes being transcribed, geotagged, and automatically linked to the corresponding evidentiary record.

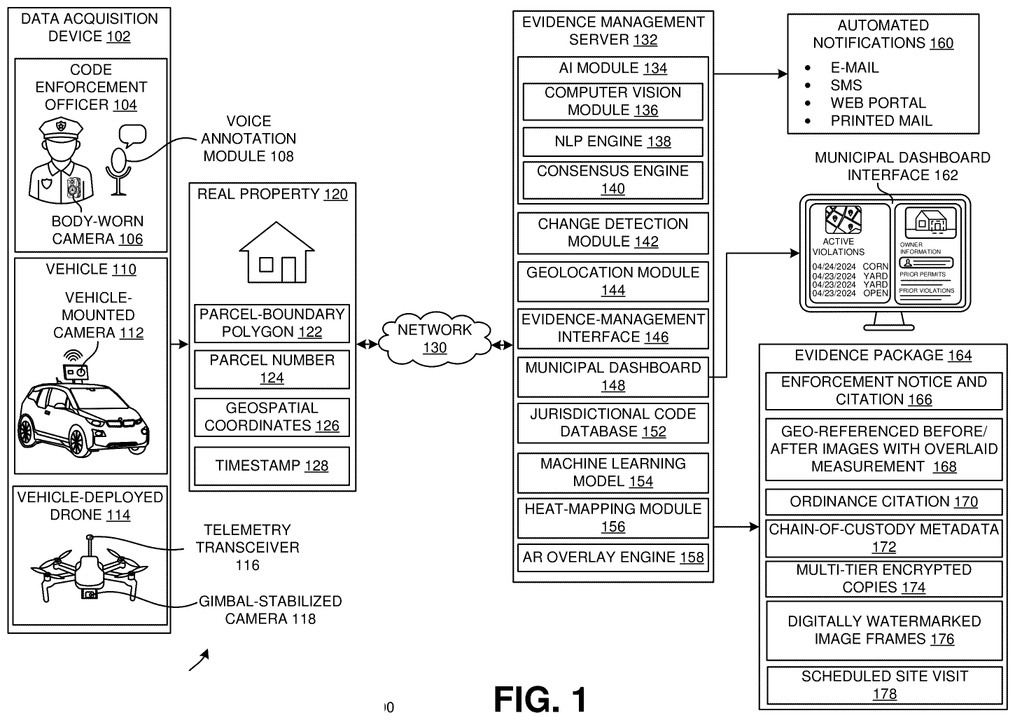

FIG. 1 is a system architecture view 150 of an automated code enforcement system 100 illustrating the components forming the system, according to one embodiment. FIG. 1, the automated code enforcement system 100 comprises a data acquisition device 102, a code enforcement officer 104, a body-worn camera 106, a voice annotation module 108, a vehicle 110, a vehicle-mounted camera 112, a vehicle-deployed drone 114, a telemetry transceiver 116, a gimbal-stabilized camera 118, a real property 120, a parcel-boundary polygon 122, a parcel number 124, a geospatial coordinates 126, a timestamp 128, a network 130, an evidence management server 132, an AI module 134, a computer vision module 136, a natural language processing (NLP) engine 138, a consensus engine 140, a change detection module 142, a geolocation module 144, an evidence-management interface 146, a municipal dashboard 148, a jurisdictional code database 152, a machine learning model 154, a heat-mapping module 156, an augmented reality (AR) overlay engine 158, an automated notification 160, a municipal dashboard interface 162, an evidence package 164, an enforcement notice and citation 166, geo-referenced before/after images with overlaid measurement 168, an ordinance citations 170, a chain-of-custody metadata 172, a multi-tier encrypted copies 174, a digitally watermarked image frames 176, and a scheduled site-visit 178, according to one embodiment.

The automated code enforcement system 100 may be an AI-powered, network-connected platform designed to help municipalities monitor, detect, and document property code compliance issues efficiently and transparently. The automated code enforcement system 100 may be defined as an integrated computational framework that combines the field-based data acquisition devices 102, such as cameras, drones, and voice annotation tools, with back-end analytical engines, including the AI module 134, the computer vision module 136, the natural language processing engine 138, the change detection module 142, and geolocation validation, all working in conjunction with geospatial databases and jurisdictional code records.

The automated code enforcement system 100 may collect images, videos, audio notes, and/or location metadata from the field via the acquisition devices 102, transmit them through the secure network 130, and process them on the evidence management server 132. The AI module 134 may orchestrate the analysis of these multimodal inputs to identify unpermitted changes and/or code violations, cross-reference them with the jurisdictional code database 152, and generate structured outputs such as the evidence packages 164, the enforcement notice and citations 166, geo-referenced before/after images with overlaid measurements 168, and the automated notifications 158. These outputs may be delivered through the municipal dashboard interface 162, enabling municipalities to streamline inspections, enforce compliance more accurately, and/or maintain an auditable trail of evidence with reduced manual intervention. The automated notifications 160 may include delivery via electronic mail, short message service, municipal web portals, and/or printed mail to alert the code enforcement officers 104 and/or municipal staff of detected violations in real time, according to one embodiment.

The data acquisition devices 102 may be a collection of field-deployed hardware components configured to capture multimedia and geospatial data, along with contextual audio information, during municipal inspections and/or monitoring activities. The data acquisition devices 102 may include a combination of wearable, vehicle-mounted, and/or aerial technologies that may gather real-time evidence of the real property 120 conditions. The data acquisition devices 102 may be equipped with the body-worn camera 106 to record ground-level images and/or video, as well as the voice annotation module 108 that may enable the code enforcement officer 104 to provide spoken notes and descriptions synchronized with the captured visual evidence. The data acquisition devices 102 may further include the vehicle 110 outfitted with the vehicle-mounted camera 112 to collect drive-by imagery during patrols, and the vehicle-deployed drone 114 equipped with the telemetry transceiver 116 and the gimbal-stabilized camera 118 to capture aerial imagery of parcels, structures, and/or surrounding areas from elevated perspectives. In some embodiments, the body-worn camera 106 and the vehicle-mounted camera 112 may operate in a synchronized mode to capture time-aligned frames of the real property 120 from multiple perspectives, which may enable frame-level correlation during analysis, according to one embodiment.

Within the automated code enforcement system 100, the data acquisition devices 102 may serve as the primary input layer, which may transmit captured multimedia evidence along with the geospatial coordinates 126, the timestamp 128, and the voice annotations via the voice annotation module 108 through the network 130 to the evidence management server 132, where the AI-module 134 may analyze the combined data to detect potential code violations and generate actionable insights to support municipal enforcement workflows.

The code enforcement officer 104 may be a municipal field agent who operates the data acquisition devices 102 to collect visual, audio, and/or geospatial data during inspections. The code enforcement officer 104 may use the body-worn camera 106 to capture ground-level photos and/or videos, and the voice annotation module 108 to record spoken notes that may be processed by the NLP engine 138 within the AI module 134 for automated transcription and tagging. The visual data from the body-worn camera 106 may be transmitted through the network 130 to the evidence management server 132, where the computer vision module 136 within the AI module 134 may analyze the imagery to detect structural changes, assess property conditions, and support automated identification of potential code violations, according to one embodiment.

The voice annotation module 108 may be an integral component of the data acquisition devices 102 designed to capture audio-based contextual information during field inspections. The voice annotation module 108 may allow the code enforcement officer 104 to record spoken notes, observations, and/or descriptive details that may be synchronized with the images and/or video collected by the body-worn camera 106 and/or other imaging devices. This capability may reduce the need for manual text entry in the field and may ensure that critical situational details, such as observed structural changes, potential hazards, and/or unique site conditions, are accurately documented in real time. Within the automated code enforcement system 100, the voice annotation module 108 may transmit the captured audio data through the network 130 to the evidence management server 132, where the NLP engine 138 may process and convert the audio into searchable, structured text for use in case files, the evidence packages 164, and/or automated violation reports. By pairing voice-based input with multimedia evidence, the voice annotation module 108 may enhance the precision, completeness, and/or efficiency of field data acquisition for municipal code enforcement workflows, according to one embodiment.

The vehicle 110 may be a transportation unit used by the code enforcement officer 104 during patrols and may be equipped with the vehicle-mounted camera 112 capable of recording exterior conditions of the real properties 120 as the vehicle 110 passes by. The vehicle 110 may be a municipal and/or enforcement vehicle that supports the operation of the automated code-enforcement system 100. The vehicle 110 may serve as a mobile platform for the data-acquisition devices 102, providing power and the network 130 connectivity for transmitting imagery and telemetry to the evidence-management server 132, where the AI module 134 may process the data for automated analysis, according to one embodiment.

The vehicle-mounted camera 112 may be mounted on the vehicle 110 to capture ground-level photos and/or videos of the real property 120 as the vehicle 110 travels near an inspection site. The imagery collected by the vehicle-mounted camera 112 may be transmitted via the network 130 to the evidence-management server 132, where the computer-vision module 136 within the AI module 134 may analyze the visual data to detect structural changes, identify unpermitted work, and/or assess property conditions relevant to jurisdictional codes, according to one embodiment.

The vehicle-deployed drone 114 may be stored in and/or on the vehicle 110, which may be launched to capture aerial and/or oblique-angle imagery of the real property 120. The vehicle-deployed drone 114 may be used to observe rooftops, rear yards, and/or other areas not visible from ground level. The collected aerial imagery may be streamed in real time to the evidence-management server 132 for processing by the AI module 134, which may use the imagery for change-detection, parcel mapping, volumetric measurement, and/or other automated analytics, according to one embodiment.

The vehicle-deployed drone 114 and/or other data-acquisition devices 102 may include a multispectral imaging module configured to capture non-visible spectral data such as infrared imagery, thermal imagery, and/or LiDAR point-cloud data. The multispectral imaging module may capture information about surface temperature variations, material composition, and elevation contours of the real property 120. This information may help the AI module 134 detect conditions not visible in standard RGB imagery, including roof degradation, heat leakage, moisture intrusion, and energy-code-related anomalies. The multispectral data may be transmitted together with the geospatial coordinates 126 and the timestamp 128 to the evidence-management server 132, where the data may be fused with conventional imagery for analysis of structural and environmental conditions associated with potential code violations, according to one embodiment.

The telemetry transceiver 116 may enable wireless communication among the vehicle-deployed drone 114, the vehicle-mounted camera 112, and the evidence-management server 132. The telemetry transceiver 116 may transmit flight metrics, location data, and/or sensor status to maintain alignment between captured imagery and the geospatial coordinates 126. The telemetry transceiver 116 data may be utilized by the AI module 134 to synchronize data streams and improve the accuracy of georeferenced analytics, according to one embodiment.

The gimbal-stabilized camera 118 may be mounted on the vehicle-deployed drone 114 and/or other aerial platform. The gimbal stabilization may allow the gimbal-stabilized camera 118 to maintain a steady orientation during flight, thereby capturing clear and stable images and/or video despite drone movement and/or wind conditions. The stabilized imagery may enhance the performance of the computer-vision module 136 within the AI module 134, improving the precision of automated detection of structural and/or environmental changes, according to one embodiment.

The real property 120 may refer to a parcel of land and/or any fixed structures built upon it, such as a house, garage, fence, and/or driveway, while excluding movable items such as vehicles and/or outdoor furniture. In the automated code-enforcement system 100, the real property 120 may serve as the subject of inspection and/or monitoring. The automated code-enforcement system 100 may identify each real property 120 using the parcel-boundary polygon 122, which may outline the legally recorded property limits within a geospatial map layer, and may link the parcel to its parcel number 124 stored in municipal records. Each captured image, the geospatial coordinate 126, and the timestamp 128 may be associated with the real property 120 to provide precise location and time-based context. This association may enable the automated code-enforcement system 100 to detect modifications to the land and/or structures, determine potential violations of jurisdictional codes, and/or generate accurate reports to support enforcement actions, according to one embodiment.

The parcel-boundary polygon 122 may define the legally recorded boundaries of the real property 120. The parcel-boundary polygon 122 may be generated from cadastral and/or municipal GIS data, which may be displayed as an outlined shape on a geospatial map layer. The automated code-enforcement system 100 may reference the parcel-boundary polygon 122 to visually separate the subject parcel from adjacent properties, support accurate geolocation of captured imagery, and guide the automated change-detection and reporting processes, according to one embodiment.

The parcel number 124 may serve as a unique municipal identifier assigned to the real property 120. The parcel number 124 may link the parcel to official municipal records, such as property ownership, permits, tax information, and/or historical inspection data. The automated code-enforcement system 100 may use the parcel number 124 to retrieve related jurisdictional data, correlate newly captured evidence with existing records, and generate reports and enforcement notices tied to the correct real property 120, according to one embodiment.

The geospatial coordinates 126 may represent the precise latitude-longitude and/or other spatial reference points that describe the physical location of the real property 120 and/or specific structures within it. The automated code-enforcement system 100 may associate the geospatial coordinates 126 with each captured image, video frame, and/or sensor reading to ensure accurate mapping within the parcel-boundary polygon 122. The geospatial coordinates 126 may enable location-specific comparison of current imagery to historical data for automated detection of structural or environmental changes, according to one embodiment.

The timestamp 128 may denote the date and time associated with each captured image, video recording, and/or sensor event collected by the data-acquisition devices 102. The timestamp 128 may be applied automatically during capture and may be stored with the geospatial coordinates 126 to provide chronological context. The automated code-enforcement system 100 may use the timestamp 128 to order inspection events, compare new imagery against prior data captured at earlier timestamps, and maintain a verifiable record that supports historical analysis, change-tracking, and evidence chain-of-custody requirements.

The network 130 may be a digital communication framework enabling the transmission of collected multimedia evidence, metadata, and geolocation information from the data acquisition devices 102 to the evidence management server 132. The network 130 may include one or more wireless communication protocols, cellular connections, local wireless networks, and/or secure cloud communication channels to maintain continuous and/or periodic synchronization between field devices and centralized analytical services, according to one embodiment.

The evidence management server 132 may be a centralized processing hub within the automated code enforcement system 100, designed to receive, store, and/or analyze multimedia and geospatial data collected by the data acquisition devices 102. The evidence management server 132 may host AI-driven analytical components, including the AI module 134, the computer vision module 136, the NLP engine 138, the consensus engine 140, the change detection module 142, and the geolocation module 144, which may work together to process incoming images, videos, and/or voice annotations. The evidence management server 132 may organize all incoming evidence into structured digital records and may link it with parcel information from the jurisdictional code database 152 for property-specific case management. The evidence management server 132 may also communicate with the municipal dashboard 148 to display analyzed outputs and generate reports, enabling automated detection of potential violations and efficient management of enforcement actions, according to one embodiment.

The AI module 134 may be a core analytical component of the evidence management server 132, configured to process multimodal field data, including video, aerial imagery, geospatial coordinates, and voice annotations received from the data acquisition devices 102. The AI module 134 may integrate the computer vision module 136 to analyze imagery and video for detecting structural changes and/or landscaping modifications, the NLP engine 138 to transcribe and tag spoken notes captured by the voice annotation module 108, the consensus engine 140 to fuse outputs from multiple trained detection models and apply confidence thresholds, the change detection module 142 to compare time-stamped imagery with historical data, and the geolocation module 144 to map detected changes to the parcel-boundary polygons 122.

The AI module 134 may further be configured to generate real-time alerts to the code enforcement officer 104 upon detection of the violation and to coordinate with the evidence-management interface 146 to produce automated enforcement notices, geo-referenced imagery, and ordinance citations.

The AI module 134 may also incorporate the machine-learning model 154 trained on historical enforcement outcomes and periodically retrained using labeled compliant and non-compliant events to improve detection accuracy over time, according to one embodiment.

The computer-vision module 136 may analyze images and video captured by the body-worn camera 106, the vehicle-mounted camera 112, and the vehicle-deployed drone 114. The computer-vision module 136 may perform automated object detection, structural-change recognition, segmentation, and other visual analytics. These outputs may be used by the AI module 134 to identify unpermitted construction, debris accumulation, or other visible conditions relevant to code enforcement, according to one embodiment.

The NLP (natural-language-processing) engine 138 may analyze text and/or spoken inputs collected through the voice-annotation module 108, written inspection notes, and/or resident-submitted reports. The NLP engine 138 may convert voice recordings into searchable text, tag key phrases, and/or match descriptions to relevant municipal code sections. These processed annotations may be stored in the evidence-management server 132 and linked to the associated real property 120.

The consensus engine 140 may aggregate outputs from the computer-vision module 136, the NLP engine 138, and the change-detection module 142 to improve the reliability of automated findings. The consensus engine 140 may apply rule-based logic and/or ensemble-learning techniques to reconcile conflicting detections, reduce false positives, and/or assign confidence scores to flagged violations.

The change detection module 142 may be a sub-component of the AI module 134 configured to compare current video frames, aerial images, and time-stamped historical footage of a parcel to detect physical changes. The change detection module 142 may apply at least one of semantic segmentation, pixel-wise differencing, and/or bounding-box generation to isolate modified regions in a structure and/or landscaping. It may further compute geometric measurements such as width, height, surface area, and/or footprint boundary, and may overlay these measurements directly on the analyzed imagery for visual confirmation. The change detection module 142 may work in coordination with the computer vision module 136 to identify unpermitted construction, demolition, and/or alterations, and may forward its outputs to the evidence-management interface 146 for report generation and citation preparation, according to one embodiment.

The geolocation module 144 may be part of the AI module 134 and may associate each captured image and/or video segment with its corresponding geographic location. The geolocation module 144 may use incoming geospatial coordinates from the data acquisition devices 102 to align imagery with the parcel-boundary polygon 122 stored in the jurisdictional code database 152. By mapping detected modifications to parcel-boundary polygons 122, the geolocation module 144 may ensure that any identified change and/or violation is accurately tied to the parcel number 124 and jurisdictional boundary, thereby supporting the generation of geo-referenced enforcement notices and the evidence packages 164, according to one embodiment.

The evidence-management interface 146 may be a software interface residing in the evidence management server 132 that enables automated organization and presentation of analyzed data. The evidence-management interface 146 may integrate with municipal property and permit databases to cross-reference detected changes against existing property records and prior permits associated with the parcel number 124. The evidence-management interface 146 may work with the AI module 134 to display violation alerts, generate automated reports, and/or prepare enforcement notices that include geo-referenced, time-stamped image frames and ordinance citations. The evidence-management interface 146 may serve as the primary channel for municipal staff to review AI-processed insights, track cases, and export the evidence packages 164 for official proceedings, according to one embodiment.

The municipal dashboard 148 may be a visualization and control layer accessible to authorized municipal personnel to monitor active violations, owner information, prior permits, prior violations, and/or priority rankings. The municipal dashboard 148 may present outputs from the AI module 134 in an intuitive format, showing heat-mapped violation clusters, severity levels, and risk indicators to prioritize inspection resources. The municipal dashboard 148 may integrate data from the evidence-management interface 146 to give officials a centralized view of all open cases and associated evidence, according to one embodiment.

The machine-learning model 154 may be embedded within the AI module 134 and may be trained on historical enforcement outcomes, such as cases where citations were upheld and/or dismissed, as well as labeled imagery of compliant and non-compliant parcels. The machine-learning model 154 may improve detection accuracy over time by adaptively retraining on new case outcomes, thereby refining decision thresholds and enhancing the performance of the computer vision module 136 and change detection module 142. This continuous learning loop may enable the system to adapt to evolving construction patterns, regional ordinance updates, and seasonal environmental changes, according to one embodiment.

The heat-mapping module 156 may be a geospatial analytics component configured to visualize detected violations across a jurisdictional map 704. The heat-mapping module 156 may ingest outputs from the AI module 134, including data linked to individual parcels of real property 120, and may assign severity levels and risk indicators to specific parcels and/or clusters of violations. The heat-mapping module 156 may display geographic trends and enforcement hotspots to highlight areas with repeated or high-risk non-compliance. This visualization may enable municipal teams to prioritize inspection routes, allocate resources more effectively, and address urgent violations first, according to one embodiment.

The AR overlay engine 158 may be an augmented-reality visualization component designed to superimpose AI-detected violations, relevant ordinance citations 170, and/or measurement overlays directly onto a live camera feed and/or pre-captured imagery. When linked to a display associated with the body-worn camera 106 and/or a handheld inspector device, the AR overlay engine 158 may allow the code enforcement officer 104 to view visual highlights of detected modifications, compliance notes, and dimensional indicators overlaid on the actual real-world scene of the real property 120. This capability may improve situational awareness during on-site inspections by enabling officers to quickly identify violation locations and their corresponding ordinance references without leaving the inspection view, according to one embodiment.

The automated notifications 160 may function as a communication component of the automated code-enforcement system 100. The automated notifications 160 may generate and deliver real-time alerts to authorized municipal personnel when the AI module 134 and/or the change-detection module 142 identifies a potential code violation or when new evidence is uploaded to the evidence-management server 132 for a specific real property 120. The automated notifications 160 may further provide status updates related to inspections, scheduled site visits 178, and required follow-up actions. These alerts may be delivered through email, SMS, web portals, printed mail, and/or other connected channels to ensure timely communication with relevant stakeholders, according to one embodiment.

The municipal dashboard interface 162 may provide an interactive, user-facing display that presents operational and analytical information derived from the automated code-enforcement system 100. The municipal dashboard interface 162 may display prioritized parcels, heat-map visualizations, active inspection cases, and alerts generated by the automated notifications 160, each of which may link to underlying evidence stored in the evidence-management server 132. The municipal dashboard interface 162 may allow authorized personnel to review AI-detected changes to the real property 120, track ongoing enforcement actions, and access parcel-specific reports, including evidence packages 164 and related ordinance citations 170, to support data-driven decision-making and efficient compliance management, according to one embodiment.

The evidence package 164 may be a compiled digital record generated by the evidence management server 132 that organizes all relevant inspection data associated with a detected violation at the real property 120. The evidence package 164 may include time-stamped, geo-referenced images and/or video frames captured by the body-worn camera 106 and the vehicle-deployed drone 114, together with the associated geospatial coordinates 126, the ordinance citation 170, and the chain-of-custody metadata 172. The evidence package 164 may be automatically assembled by the evidence-management interface 146 and may serve as a complete parcel-specific case file that municipal staff may review on the municipal dashboard 148, archive for recordkeeping, and/or submit for official enforcement proceedings, according to one embodiment.

The enforcement notice and citation 166 may be an automatically generated document prepared by the evidence-management interface 146 in coordination with the AI module 134. The enforcement notice and citation 166 may incorporate a detected violation's ordinance citation 170, the parcel number 124 linked to the real property 120, a description of the violation, and at least one time-stamped, geo-referenced evidentiary image frame captured by the data acquisition devices 102. The enforcement notice and citation 166 may be configured for delivery through the automated notifications 160 to property owners or municipal staff and/or may be included as part of the evidence package 164 to provide a legally compliant, ready-to-issue enforcement document. By associating each enforcement notice with the corresponding real property and its parcel-specific evidence, the system may streamline official compliance actions and maintain proper legal traceability, according to one embodiment.

The geo-referenced before/after images with overlaid measurement 168 may include comparative image frames of the real property 120 that are captured at different timestamps 128 and aligned with the geospatial coordinates 126 of the parcel. These comparative images may be generated by the change-detection module 142 to highlight modifications to a physical structure and/or landscaping located within the boundaries of the parcel-boundary polygon 122. The overlaid measurements may consist of analytical graphics applied by the change-detection module 142 to display quantitative dimensions, such as width, height, surface area, and/or footprint boundary, directly on the imagery and/or video frames of the real property 120. These combined overlays may assist code enforcement officers 102 in confirming the scale and location of detected changes, such as a new fence, driveway, addition, and/or accessory structure built on the parcel. The geo-referenced before/after images with overlaid measurement 168 may be stored in the evidence package 164 and/or presented on the municipal dashboard 148 to provide clear visual proof and precise dimensional context for a detected violation associated with the real property 120, according to one embodiment.

The ordinance citation 170 may reference one or more sections of digitized municipal regulations that are relevant to a detected violation associated with the real property 120. The ordinance citation 170 may be automatically generated and mapped by the NLP engine 138 within the AI module 134 to connect the detected modification, such as unpermitted construction, landscaping changes, and/or other code-related alterations, to the corresponding ordinance clause and/or statutory requirement. The ordinance citation 170 may be displayed alongside the evidentiary imagery and geo-referenced before/after images with overlaid measurement 168 in the enforcement notice and citation 166 to provide a clear legal context for municipal inspectors and property owners, supporting compliance actions and documentation of the specific rule or regulation that applies, according to one embodiment.

The chain-of-custody metadata 172 may be a secure audit record automatically generated by the evidence management server 132 to log all actions performed on evidentiary data associated with the real property 120. The chain-of-custody metadata 172 may include timestamps for data capture, access history, cryptographic hashes, and/or a tamper-evident audit trail. The chain-of-custody metadata 172 may be embedded in the evidence package 164 to ensure evidentiary integrity for use in official enforcement and/or legal proceedings, according to one embodiment.

The multi-tier encrypted copies 174 may be encrypted versions of sensitive evidentiary imagery maintained by the evidence management server 132. The multi-tier encrypted copies 174 feature may allow the automated code enforcement system 100 to generate a redacted public copy for general access while retaining an unredacted secured copy under restricted chain-of-custody controls. The multi-tier encrypted copies 174 may help protect resident privacy for imagery captured from or near the real property 120 while meeting legal and compliance requirements for data security, according to one embodiment.

The digitally-watermarked image frames 176 may be images and/or video frames enhanced with embedded metadata such as the parcel number 124, the ordinance citation 170, the timestamp 128, and/or the geospatial coordinates 126. This watermarking may be applied by the evidence management server 132 to ensure authenticity and prevent tampering of evidence associated with the real property 120. The digitally-watermarked image frames 176 may be stored within the evidence package 164 and presented in the enforcement notice and citation 166 to maintain verifiable traceability of the parcel-specific evidence, according to one embodiment.

The scheduled site visit 178 may be a component included within the evidence package 164 to assist municipal staff in planning and coordinating follow-up inspections at the real property 120. The scheduled site visit 178 may work in connection with the municipal dashboard 148 to display inspection schedules, generate automated reminders, and prioritize site visits based on violation severity, location risk, and/or urgency. By including the scheduled site-visit 178 as part of the evidence package 164, the automated code enforcement system 100 may streamline inspection workflows, reduce delays, and improve operational efficiency for municipal enforcement teams, according to one embodiment.

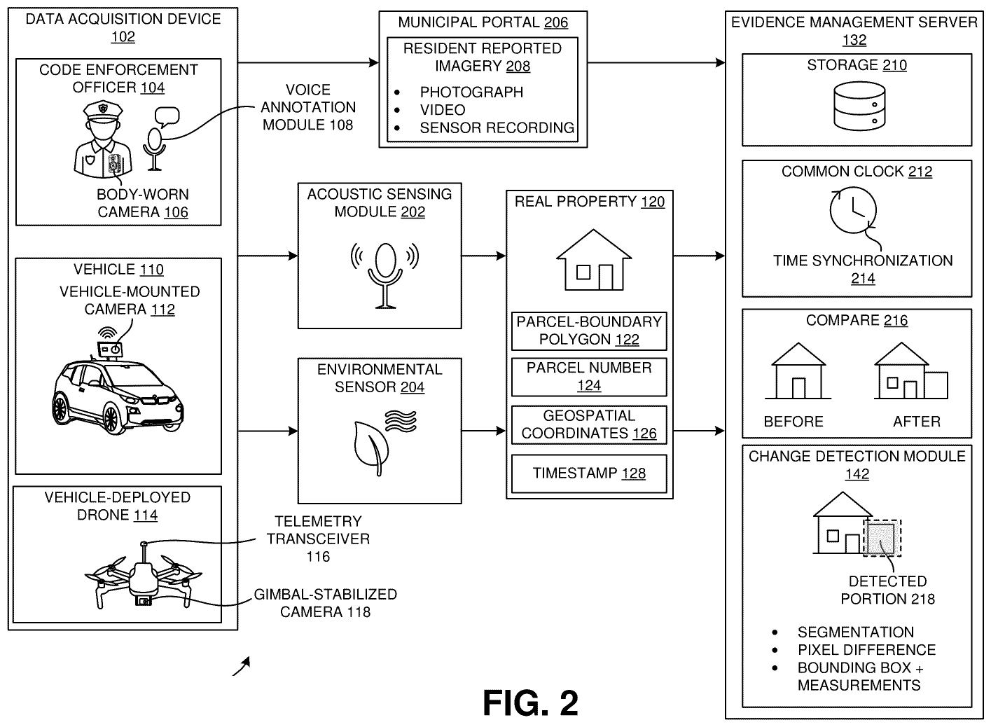

FIG. 2 is a sensor integration and change detection view 250 of the automated code enforcement system 100, according to one embodiment. FIG. 2 illustrates the data acquisition device 102, the code enforcement officer 104, the body-worn camera 106, the voice annotation module 108, the vehicle 110, the vehicle-mounted camera 112, the vehicle-deployed drone 114, the telemetry transceiver 116, the gimbal-stabilized camera 118, the evidence management server 132, an acoustic sensing module 202, an environmental sensor 204, a municipal portal 206, a resident-reported imagery 208, a storage 210, a common clock 212, a time synchronization 214, a compare module 216, a change detection module 218, and a detected portion 220, according to one embodiment.

The acoustic sensing module 202 may be an auxiliary sensor unit configured to capture audio signatures associated with activities occurring at or near the real property 120. The acoustic sensing module 202 may detect non-compliant sound patterns such as construction machinery operating outside permitted hours, amplified sound levels exceeding municipal noise ordinances, and/or unpermitted industrial equipment. The acoustic sensing module 202 may transmit time-stamped audio data to the evidence management server 132 through the network 130, where it may be synchronized with visual and geospatial inputs using the common clock 212. The AI module 134 within the evidence management server 132 may analyze the audio stream using pattern-recognition techniques to classify detected sound events, assign a confidence level to each detected sound, and correlate the classified audio events with geo-referenced video data for inclusion in violation detection workflows and/or evidence packages 164, according to one embodiment.

The environmental sensor 204 may be a field-deployed sensing unit configured to capture ambient environmental parameters that may indicate potential code violations at the real property 120. The environmental sensor 204 may detect conditions such as air-quality degradation, dust generation from construction, light emissions, and/or improper waste disposal linked to environmental regulations. The environmental sensor 204 may transmit its measurements together with geospatial coordinates 126 and timestamps 128 to the evidence management server 132, where the readings may be synchronized with other sensor inputs using the common clock 212. The AI module 134 may fuse the environmental readings with imagery and audio inputs to identify patterns suggestive of non-compliance and may cross-reference these detections with applicable environmental regulations stored in the digitized code database 302 to determine compliance status. The AI-driven analysis may strengthen the correlation between physical evidence and environmental impacts, thereby supporting more accurate violation classification for the associated parcel number 124, according to one embodiment.

The municipal portal 206 may be a digital reporting interface configured to allow residents to submit evidentiary inputs directly to the automated code-enforcement system 100. The resident-reported imagery 208 may include photographs, video clips, and/or sensor recordings documenting potential violations at the real property 120. These submissions may be transmitted through the municipal portal 206 to the evidence management server 132, where they may be time-stamped, geo-tagged, and synchronized with official sensor and camera inputs using the common clock 212, according to one embodiment.

The AI module 134 may process the resident-reported imagery 208 alongside acoustic and environmental data to assess consistency, assign confidence scores, and incorporate corroborated evidence into violation detection workflows and the evidence packages 164. The municipal portal 206 may be accessible to residents solely for submitting evidentiary inputs; submitted data may not be visible to the public but may be routed to the evidence-management server 132 and made available only to authorized municipal staff through the evidence-management interface 146 and/or the municipal dashboard interface 162 for official review and enforcement purposes, according to one embodiment.

The common clock 212 may be a centralized timing mechanism within the evidence management server 132 that distributes a synchronized reference signal to the body-worn camera 106, the vehicle-mounted camera 112, the vehicle-deployed drone 114, and supplemental sensors such as the acoustic sensing module 202 and the environmental sensor 204. The time-synchronization 214 process may ensure that video frames, audio samples, and environmental readings align precisely with one another at the same capture intervals, enabling the AI module 134 and its change-detection module 142 to perform frame-level correlation across multi-perspective data streams. This synchronized dataset may be used by the compare module 216 to align “before” and “after” imagery and by the change-detection module 142 to identify potential modifications in physical structures and/or site conditions, according to one embodiment.

The compare module 216 may be a software component of the evidence management server 132 configured to align current time-synchronized video data with imagery associated with an immediately previous timestamp 128 for the same parcel number 124. The AI module 134 may use the compare module 216 to generate paired “before” and “after” image frames for analysis and to produce intermediate difference maps that highlight regions of detected change. These aligned image pairs may then be forwarded to the change-detection module 142, where the AI-driven analysis may apply geometric measurements such as width, height, and/or footprint boundaries to classify and document detected modifications as potential violations, according to one embodiment.

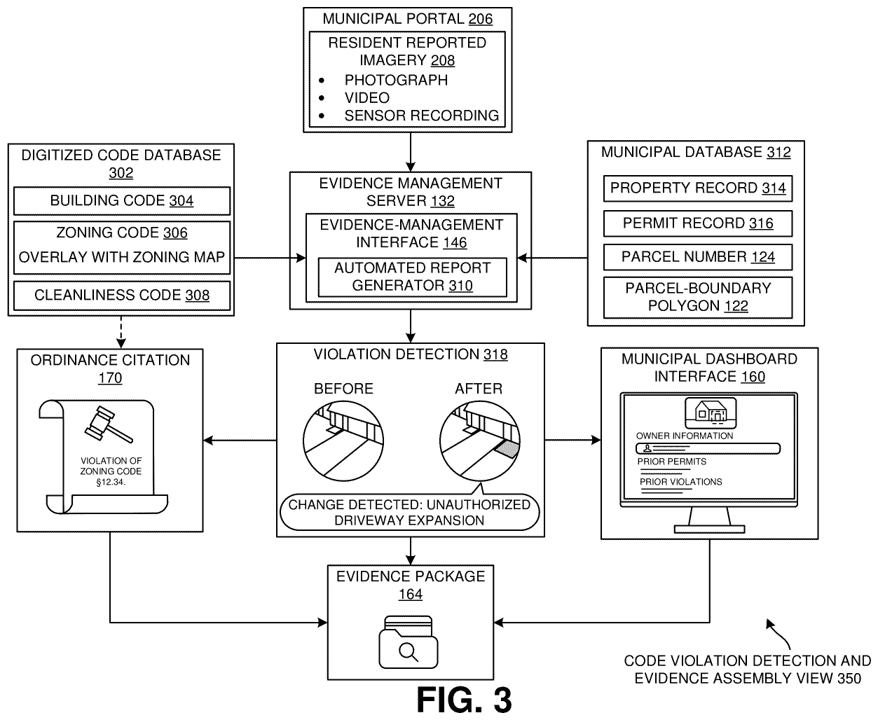

FIG. 3 is a code violation detection and evidence assembly view 350 of the automated code enforcement system 100, according to one embodiment. FIG. 3 illustrates the parcel-boundary polygon 122, the parcel number 124, the evidence management server 132, the evidence-management interface 146, the municipal dashboard interface 162, the evidence package 164, the ordinance citation 170, the municipal portal 206, the resident-reported imagery 208, a digitized code database 302, a building code 304, a zoning code 306, a cleanliness code 308, a automated report generator 310, a municipal database 312, a property record 314, a permit record 316, and a violation detection 318, according to one embodiment.

The digitized code database 302 may be a structured digital repository of jurisdictional regulations accessible by the evidence-management server 132 and the AI module 134. The database 302 may include, for example, the building code 304, the zoning code 306, and the cleanliness code 308, among other municipal and/or statutory regulations. The digitized code database 302 may enable the automated code-enforcement system 100 to automatically cross-reference detected structural and/or landscaping modifications at the real property 120 with one or more relevant ordinance provisions to determine whether a violation of a jurisdictional regulation has occurred. The AI module 134 may query the digitized code database 302 to identify applicable rules for each detected change. The ordinance citation 170 may be derived directly from the digitized code database 302 to ensure that every detected violation is tied to an authoritative regulation section, according to one embodiment.

The building code 304 may be a digitized collection of municipal building regulations defining construction standards, structural-safety requirements, and/or guidelines for permitted alterations. The AI module 134 and the evidence-management interface 146 may query the building code 304 to verify whether a detected portion 216, such as an added structure, expansion, and/or alteration at the real property 120, complies with construction rules and/or represents a violation, according to one embodiment.

The zoning code 306 may be a digitized set of land-use regulations defining allowable uses of parcels, lot-coverage limits, setbacks, and permitted accessory structures. The evidence-management server 132, in coordination with the AI module 134, may overlay detected parcel modifications onto zoning-map layers to identify mismatches between actual property use and designated zoning classifications. Detected mismatches may be automatically flagged and included in the enforcement notice and citation 170, which may be visually overlaid on zoning-map layers within the municipal dashboard 148 for review by the code-enforcement officer 104. The AI module 134 may further analyze parcel changes against the zoning code 306 to identify non-compliant activities such as driveway expansions, fencing modifications, and/or building-footprint increases that exceed zoning limits, according to one embodiment.

The cleanliness code 308 may be a digitized repository of municipal standards related to sanitation, waste management, and visual-blight regulations. The evidence-management server 132 and/or the AI module 134 may use the cleanliness code 308 to evaluate whether detected site conditions at the real property 120, such as debris accumulation, littering, and/or improper waste storage, constitute violations requiring enforcement. Cleanliness-related detections may be included in the ordinance citation 170, may be cross-referenced with the digitized code database 302, and may be displayed as alerts on the municipal dashboard interface 162, according to one embodiment.

The automated report generator 310 may be a software component of the evidence-management server 132 configured to prepare structured outputs, including violation notices, ordinance citations, and/or formatted municipal reports. The automated report generator 310 may automatically collect parcel details for the real property 120, apply relevant ordinance citations, and embed evidentiary imagery generated by the AI module 134 to create reports that comply with municipal enforcement procedures, according to one embodiment.

The municipal database 312 may store official property-related data that is accessible by the evidence-management server 132. The municipal dashboard 148 may display active violations together with owner information, prior permits, and/or violation history, enabling municipal staff to review parcel-specific enforcement history at a glance on the municipal dashboard interface 162. The municipal database 312 may include the property record 314 describing parcel ownership and address information, the permit record 316 that logs historical approvals for construction and/or landscaping activities, and geospatial identifiers such as the parcel number 124 and the parcel-boundary polygon 122 to enable precise mapping of detected changes at the real property 120, according to one embodiment.

The property record 314 may be a digitized repository of official parcel-specific details maintained within the municipal database 312. It may include the parcel owner's name, mailing address, contact information, parcel address, land-use designation, and/or historical usage data. The property record 314 may also maintain links to prior inspection notes, violation history, and/or recorded changes to the parcel-boundary polygon 122. The evidence-management interface 146 may access the property record 314 whenever a new violation is detected to associate the evidence with the correct parcel number 124 and/or the responsible owner. By mapping the detected change to the parcel number 124 and the parcel-boundary polygon 122, the automated code-enforcement system 100 may automatically connect the violation data to the relevant property record 314 for case creation. This linkage may ensure that all subsequent enforcement notices, the ordinance citations 170, and/or scheduled site visits 178 are accurately addressed to the appropriate property owner. Including the property record 314 within the workflow may allow municipal staff to review ownership details, prior permits, and historical compliance patterns directly from the municipal dashboard 148, improving traceability and legal validity in enforcement actions, according to one embodiment.

The permit record 316 may be a structured, digitized log of construction, alteration, and/or landscaping permits issued for each real property 120. It may contain permit-application data such as approval dates, expiration dates, scope of work, contractor details, inspection outcomes, and/or status (e.g., active, expired, or revoked). The AI module 134 may query the permit record 316 during violation detection to verify whether a newly identified modification, such as a footprint expansion, structural addition, and/or landscaping change at the real property 120, was previously authorized. If the queried permit data does not show approval for the detected modification, the automated code-enforcement system 100 may classify the change as unpermitted and flag it for enforcement review, according to one embodiment.

The permit record 316 may further support automated comparison by the change-detection module 142, the AI module 134, and/or the evidence-management interface 146, allowing the code-enforcement officer 104 to quickly determine whether the detected modification matches an approved permit and/or represents a potential violation. Integrating the permit record 316 with the evidence package 164 may strengthen the audit trail by including direct references to the relevant permit history, thereby enhancing transparency and legal compliance in municipal code-enforcement workflows, according to one embodiment.

The violation detection 318 may represent the automated code-enforcement system 100's capability to compare “before” and/or “after” images and/or video frames to identify physical modifications on the real property 120. The violation detection 318 may rely on outputs from the change-detection module 142 and the AI module 134 to analyze differences and confirm whether a structural, zoning, and/or cleanliness-related change breaches applicable municipal codes. The AI module 134 may further correlate the detected changes with the digitized code database 302 to automatically identify the relevant ordinance citation 170 and generate a system-assigned confidence score for the violation classification. Each violation instance may be associated with a system-generated confidence percentage, providing inspectors with a quantitative reliability indicator. In some embodiments, the evidence-management server 132 may also receive resident-reported imagery 208, including photographs, video recordings, and/or sensor submissions uploaded via the municipal portal 206, and the AI module 134 may fuse this resident input with system-captured evidence to corroborate violation detection, according to one embodiment.

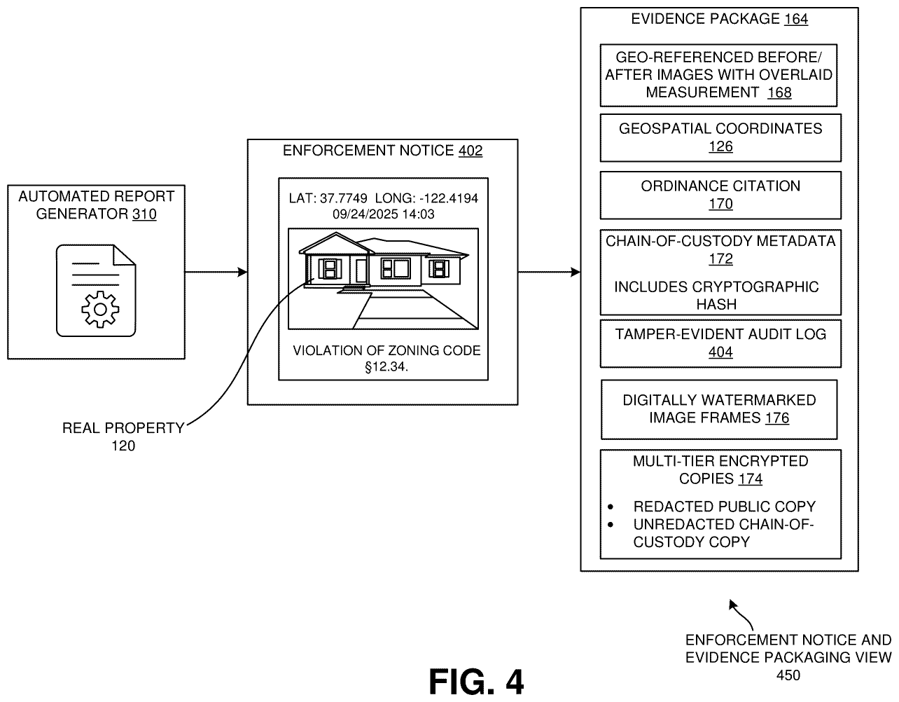

FIG. 4 is an enforcement notice and evidence packaging view 450 of the automated code enforcement system 100, according to one embodiment. FIG. 4 illustrates the geospatial coordinates 128, the evidence package 164, the ordinance citation 170, the chain-of-custody metadata 172, the multi-tier encrypted copies 174, the digitally watermarked image frames 176, the automated report generator 310, an enforcement notice 402, and a tamper-evident audit log 404, according to one embodiment.

The automated report generator 310 may prepare the structured enforcement notice 402 that includes key violation details of the real property 120. The enforcement notice 402 may contain a representative image of the real property 120 with the detected modification highlighted, along with the geospatial coordinates 128, such as latitude, longitude, and/or timestamp, to identify where and when the violation was recorded. The enforcement notice 402 may further include the ordinance citation 170 referencing the applicable regulatory section, thereby providing a legally grounded basis for the enforcement action, according to one embodiment.

Once created, the enforcement notice 402 may be transmitted into the evidence package 164. The evidence package 164 may serve as a consolidated case file that supports municipal enforcement workflows and legal proceedings. This evidence package 164 may include geo-referenced before/after images with overlaid measurement 168 to highlight changes in structures and/or landscaping, verified geospatial coordinates 128, and the ordinance citation 170. The evidence package 164 may also include a structured metadata record identifying the source of the imagery, sensor calibration details, and verification notes from human reviewers, thereby strengthening admissibility in administrative hearings and/or judicial proceedings, according to one embodiment.

Complementing this, the tamper-evident audit log 404 may serve as an immutable ledger that records each interaction with the evidentiary data, preventing unauthorized modifications. The tamper-evident audit log 404 may be stored in a blockchain-backed and/or similarly distributed system to ensure immutability and verifiability. The tamper-evident audit log 404 may capture the automated code enforcement system 100 events, such as report generation, evidence packaging, reviewer annotations, and/or official transmittal of the enforcement notice 402, according to one embodiment.

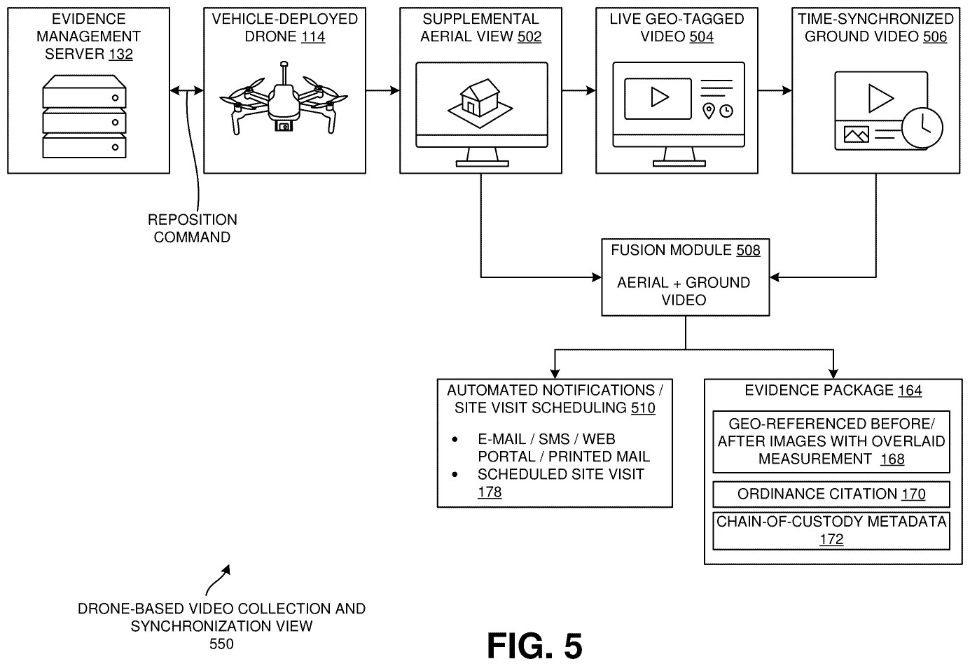

FIG. 5 is a drone-based video collection and synchronization view 550 of the automated code enforcement system 100, according to one embodiment. FIG. 5 illustrates the vehicle-deployed drone 114, the evidence management server 132, the evidence package 164, the geo-referenced before/after images with overlaid measurement 168, the ordinance citation 170, the chain-of-custody metadata 172, a supplemental aerial view 502, a live geo-tagged video 504, a time-synchronized ground video 506, a fusion module 508, a automated notifications and site visit scheduling 510, according to one embodiment.

The evidence management server 132 may be a centralized computational hub that communicates with the vehicle-deployed drone 114 to initiate aerial data capture. The evidence management server 132 may issue commands to reposition the vehicle-deployed drone 114 for better coverage of a detected portion of the real property 120, as needed for verification and/or supplemental evidence gathering. The vehicle-deployed drone 114 may be equipped with the gimbal-stabilized camera 118 and the telemetry transceiver 116 to capture high-resolution aerial video and transmit it back to the evidence management server 132 in real time. The vehicle-deployed drone 114 may be launched from the vehicle 110 and may provide additional visual perspectives that complement the body-worn camera 106 and the vehicle-mounted camera 112, according to one embodiment.

The supplemental aerial view 502 may represent an enhanced aerial perspective generated by the vehicle-deployed drone 114. The supplemental aerial view 502 may capture specific angles and/or areas of interest, such as rooftops, backyards, and/or other zones not visible from street-level ground cameras. The supplemental aerial view 502 may be especially useful for documenting structural additions, landscaping changes, and/or volumetric modifications on the real property 120, according to one embodiment.

The live geo-tagged video 504 may be a real-time streaming video feed transmitted from the vehicle-deployed drone 114 to the evidence management server 132. This live geo-tagged video 504 may include the geospatial coordinates 126 and the timestamps 128, allowing every video frame to be tied to a precise location and moment of capture. The live geo-tagged video 504 may be accessible to the code enforcement officers 104 and authorized municipal staff through the municipal dashboard interface 162, enabling them to view aerial perspectives of a parcel as they are recorded. By providing a synchronized, location-aware stream, the live geo-tagged video 504 may allow remote validation of detected structural and/or landscaping changes without requiring immediate on-site presence. This capability may enhance situational awareness for the code enforcement officers 104 during active investigations, support quick decision-making in cases where violations must be confirmed urgently, and improve operational efficiency by reducing the need for unnecessary field visits while still maintaining accurate geospatial context for enforcement purposes, according to one embodiment.

The time-synchronized ground video 506 may be created by aligning the live aerial feed received from the vehicle-deployed drone 114 with ground-level imagery captured simultaneously by the body-worn camera 106 and/or the vehicle-mounted camera 112. The common clock 208 within the evidence management server 132 may coordinate these multi-perspective data streams to ensure that each frame from both aerial and ground views corresponds precisely to the same moment in time, according to one embodiment.

By producing a unified, synchronized video set, the automated code enforcement system 100 may enable the change-detection module 214 to analyze visual evidence from multiple angles with improved accuracy. This combined perspective may enhance the detection and measurement of structural and/or landscaping modifications, allowing the code enforcement officers 104 to validate observed changes more effectively and reducing the possibility of errors caused by unsynchronized and/or incomplete footage, according to one embodiment.

The fusion module 508 may be configured to combine aerial imagery from the supplemental aerial view 502 with synchronized ground-level video 508. This integration may enhance the precision of detection workflows, which may enable the AI module 134 to validate geospatial consistency, cross-verify changes from multiple perspectives, and strengthen volumetric measurement accuracy, according to one embodiment.

The automated notifications and site visit scheduling 510 may be a workflow component linked to the evidence management server 132. It may issue inspection-related alerts via email, SMS, web portal updates, and/or printed mail, and may further schedule follow-up site visits 178 based on violation severity and/or compliance status. This feature may streamline enforcement timelines and improve operational efficiency, according to one embodiment.

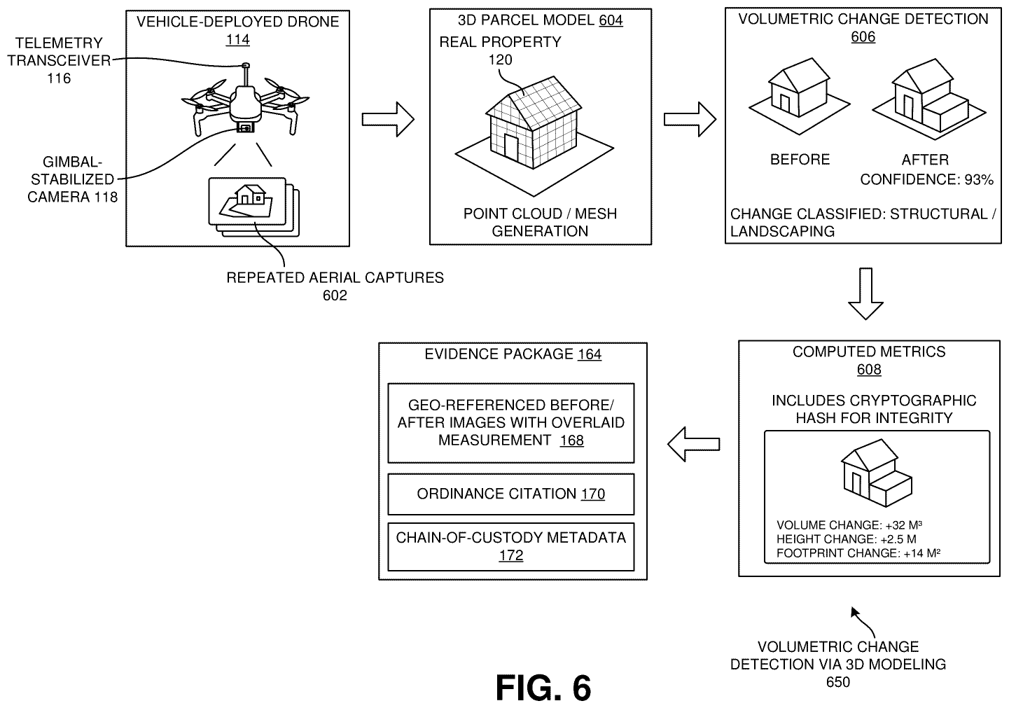

FIG. 6 is a volumetric change detection via 3D parcel modeling 650 of the automated code enforcement system 100, according to one embodiment. FIG. 6 illustrates the vehicle-deployed drone 114, the telemetry transceiver 116, the gimbal-stabilized camera 118, the real property 120, the geo-referenced before/after images with overlaid measurement 168, the ordinance citation 170, and the chain-of-custody metadata 172, the evidence package 164, a repeated aerial captures 602, a 3D parcel model 604, a volumetric change detection 606, and a computed metrics 608, according to one embodiment.

The vehicle-deployed drone 114 may be launched from a municipal vehicle and may carry the gimbal-stabilized camera 118 and the telemetry transceiver 116. The vehicle-deployed drone 114 may perform the repeated aerial captures 602 of the real property 120 at scheduled intervals and/or as triggered by the evidence management server 132. These repeated aerial captures 602 may generate overlapping image sets of the real property 120 from multiple angles to support three-dimensional reconstruction, according to one embodiment.

The 3D parcel model 604 may be a digitally reconstructed three-dimensional representation of the real property 120. The reconstruction may be generated through point cloud and/or mesh generation techniques, such as photogrammetry and/or structure-from-motion, to produce a spatially accurate model. The evidence management server 132, working in conjunction with the AI module 134, may align the new 3D parcel model 604 with a previously stored baseline model to detect structural and/or landscaping modifications, according to one embodiment.

The volumetric change detection 606 may involve comparing the current 3D parcel model 604 to the baseline model to identify dimensional differences. This process may reveal changes such as footprint expansion, height increases, roofline modifications, and/or partial demolitions. The volumetric change detection 606 may further classify changes such as structural and/or landscaping modifications, which may generate a confidence percentage (e.g., 93%) to indicate the reliability of the detection, according to one embodiment.

The computed metrics 608 may quantify the detected volumetric changes with numerical outputs such as volume change (e.g., +32 m³), height change (e.g., +2.5 m), and/or footprint change (e.g., +14 m²). Each computed metric 608 may also include a cryptographic hash to preserve evidentiary integrity and prevent tampering of measurement outputs. These computed metrics 608 may be automatically overlaid on the 3D model and/or on two-dimensional projections to aid in visualization and decision-making, according to one embodiment.

The AI module 134 may play a central role by automating feature extraction from aerial images, registering temporal models for accurate comparison, and correlating volumetric metrics with applicable municipal code provisions stored in the digitized code database 302. The outputs of volumetric change detection 606 and computed metrics 608 may be transferred into an evidence package 164, which may include the geo-referenced before/after images with overlaid measurements 168, the ordinance citation 170, and the chain-of-custody metadata 172. These packaged outputs may support the generation of violation reports and the issuance of enforcement actions by municipal staff, according to one embodiment.

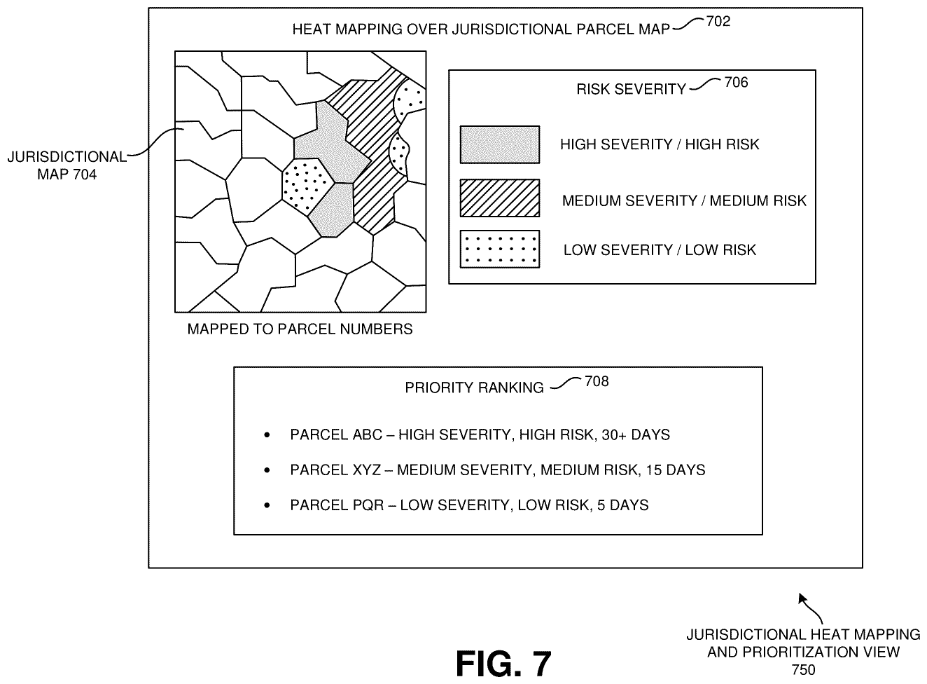

FIG. 7 is a jurisdictional heat mapping and prioritization view 750 of the automated code enforcement system 100, according to one embodiment. FIG. 7 illustrates a heat mapping over a jurisdictional parcel map 702, a jurisdictional map 704, a risk severity 706, and a priority ranking 708, according to one embodiment.

The heat mapping over the jurisdictional parcel map 702 may represent a visualization layer generated by the heat-mapping module 154 operating within the evidence management server 132. The heat mapping 702 may integrate real-time violation data with the parcel-boundary polygons 122 stored in the municipal database 312 to display spatial concentrations of detected non-compliance. Each parcel displayed on the jurisdictional map 704 may be rendered with color coding and/or pattern overlays to highlight areas where detected violations exhibit significant severity and/or risk levels. This spatial visualization may help the code enforcement officers 104 to quickly identify clusters of high-priority cases that may warrant immediate attention. The heat mapping 702 may further be mapped directly to the parcel numbers 124 so that each visualized violation is linked to a unique identifier for enforcement actions, according to one embodiment.

The jurisdictional map 704 may comprise geospatial data showing all parcels within a city and/or other designated jurisdictional boundary. The jurisdictional 704 may be dynamically updated as the automated code enforcement system 100 continuously ingests new evidence from the body-worn camera 106, the vehicle-mounted camera 112, and the vehicle-deployed drone 114, as well as supplemental inputs such as the environmental sensor 204 and/or the acoustic sensing module 202. As new violations are detected, the jurisdictional map 704 may automatically adjust its visual indicators to reflect the changing compliance status of each parcel in near real time. The jurisdictional map 704 may therefore function as the geospatial foundation upon which heat mapping 702 and risk severity 706 visualizations are layered, according to one embodiment.

The risk severity 706 legend may categorize parcels into tiers such as high severity/high risk, medium severity/medium risk, and low severity/low risk based on a multi-factor analysis conducted by the heat-mapping module 154 and supported by the AI module 134. Factors considered in determining severity and risk may include the type of violation (e.g., unauthorized structural expansion versus minor cleanliness issues), the potential safety and/or environmental impact of the violation, the parcel's historical record of prior violations, and the elapsed time since the issue was first detected. For example, a large unpermitted structure erected in a flood-risk zone may be categorized as high severity/high risk, whereas minor debris accumulation may fall into a lower-risk tier. The risk severity 706 may provide the code enforcement officers 104 with at-a-glance clarity when reviewing the jurisdictional map 704, according to one embodiment.

The priority ranking 708 may display a list of parcels ordered according to their combined severity, risk level, and the duration since detection of the violation. The priority ranking 708 may be automatically generated by the evidence-management interface 146 and may be displayed on the municipal dashboard interface 162 for actionable decision-making by enforcement personnel. For instance, a parcel ABC with a detected unpermitted addition classified as high severity/high risk and unresolved for 30+ days may be listed at the top of the ranking, while a parcel PQR with low severity/low risk detected only 5 days ago may appear lower in priority. Such a ranking may allow municipal staff to allocate limited field resources to the most urgent cases. The priority ranking 708 may also include automatically suggested inspection timelines, such as 5-day, 15-day, and/or 30-day windows, to guide scheduling, according to one embodiment.

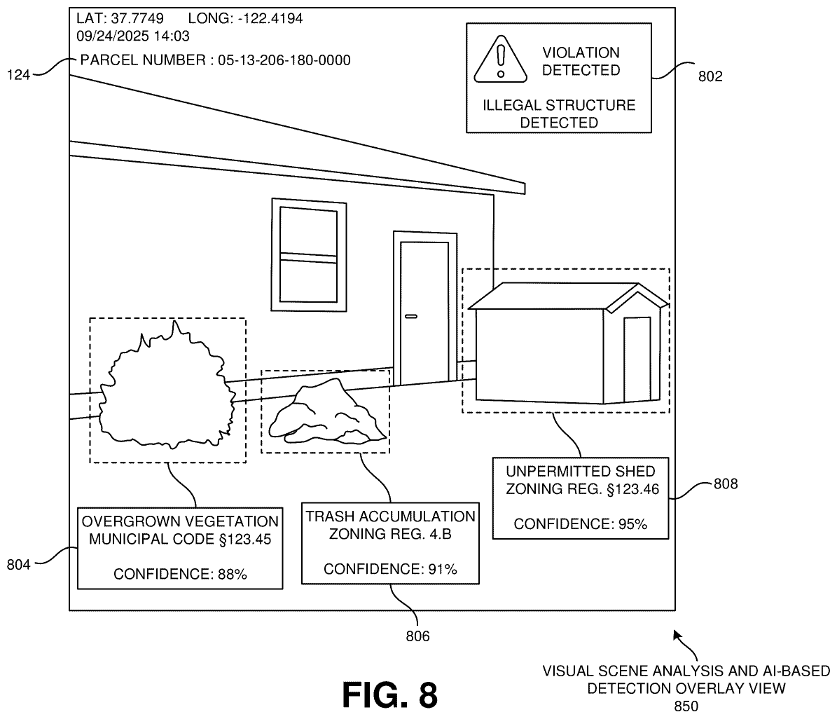

FIG. 8 is a visual scene analysis and AI-based detection overlay view 850 of the automated code enforcement system 100, according to one embodiment. FIG. 8 illustrates the parcel number 124, a violation detected indicator 802, an overgrown vegetation detection 804, a trash accumulation detection 806, and an unpermitted shed detection 808, according to one embodiment.

The figure illustrates a real-time inspection in progress. The code-enforcement officer 104 wearing the AI-equipped body-worn camera 106 arrives at the real property 120 for the scheduled site visit 178 check. As soon as the code-enforcement officer 104 activates the body-worn camera 106, it begins capturing and streaming high-definition video frames tagged with the geospatial coordinates 126, the parcel number 124, and the live timestamp 128 at the top of the screen. The geolocation module 144 on the evidence-management server 132 confirms the footage belongs to the correct real property 120 by matching the live coordinates to the parcel-boundary polygon 122. This ensures that each detection is tied to the correct parcel number 124 for enforcement purposes, according to one embodiment.

This same live video feed is transmitted over the network 130 to the municipal dashboard interface 162, where supervisors at the municipal office can view the code-enforcement officer 104's perspective in real time. As the code-enforcement officer 104 slowly scans the yard, the AI module 134 running jointly in the body-worn camera 106 and on the evidence-management server 132 begins analyzing each frame, and the computer-vision engine 136 overlays detection boxes directly on the streaming footage. Each overlay may include both a violation label and a confidence score, giving code-enforcement officers 104 and supervisors a real-time reliability indicator, according to one embodiment.

On the municipal dashboard interface 162, the first overlay appears on the left side of the screen: a bounding box 804 highlights a dense shrub, flashing a label that reads:
“Overgrown Vegetation—Municipal Code § 123.45—Confidence: 88%.”

This visual marker tells both the officer and the supervisors watching at the municipal office that the change-detection module 142 has compared the shrub's size to baseline parcel imagery and flagged it as potentially exceeding the allowed growth limit in the digitized code database 302. The violation is automatically logged in the active violations list for the parcel number 124, according to one embodiment.

The body-worn camera 106 pans toward the driveway. A second bounding box 806 lights up around a heap of discarded material. The overlay now reads:
“Trash Accumulation—Zoning Reg. 4.B—Confidence: 91%.”

The AI module 134 cross-references the zoning code 306 and finds no active permit for waste storage on this real property 120, marking it as a probable violation of municipal cleanliness regulations. The municipal dashboard interface 162 shows these alerts instantly to staff at their desks, giving them a synchronized view of the code-enforcement officer 104's findings. The evidence-management interface 146 also saves the annotated frame into the evidence package 164 with chain-of-custody metadata 172, according to one embodiment.

As the code-enforcement officer 104 shifts the body-worn camera 106 to the right side of the yard, the AI module 134 detects a detached shed structure. A bright red violation alert 802 appears on both the officer's body-cam display and the live dashboard feed:
“Violation Detected—Illegal Structure—Zoning Reg. § 123.46—Confidence: 95%.”

A large bounding box 808 outlines the shed itself. The system's change-detection module 214 recognizes that the shed's footprint and height differ from historical imagery and that no matching permit record 316 exists in the municipal database 312, leading to a real-time unpermitted-structure alert. The violation alert 802 may be prioritized for follow-up scheduling through the automated notifications 160, according to one embodiment.

Every detection for overgrown vegetation 804, for trash accumulation 806, and for the shed 808 is geo-referenced, time-stamped, and ordinance-linked at the moment of capture. The evidence-management interface 146 saves the annotated frames directly into the evidence package 164, adding the chain-of-custody metadata 172 for audit tracking. Meanwhile, the municipal dashboard interface 162 updates its active-violations list live as each new detection arrives, letting supervisors follow the inspection as if they were standing next to the code-enforcement officer 104. These annotated detections may later be compiled into the enforcement notices 402 and the ordinance citations 170 for delivery to the property owner, according to one embodiment.

Although the present embodiments have been described with reference to specific example embodiments, it will be evident that various modifications and changes may be made to these embodiments without departing from the broader spirit and scope of the various embodiments. For example, the various devices and modules described herein may be enabled and operated using hardware circuitry (e.g., CMOS based logic circuitry), firmware, software and/or any combination of hardware, firmware, and software (e.g., embodied in a non-transitory machine-readable medium). For example, the various electrical structure and methods may be embodied using transistors, logic gates, and electrical circuits (e.g., application specific integrated (ASIC) circuitry and/or Digital Signal Processor (DSP) circuitry).

In addition, it will be appreciated that the various operations, processes and methods disclosed herein may be embodied in a non-transitory machine-readable medium and/or a machine-accessible medium compatible with a data processing system (e.g., data processing device 100). Accordingly, the specification and drawings are to be regarded in an illustrative rather than a restrictive sense.

A number of embodiments have been described. Nevertheless, it will be understood that various modifications may be made without departing from the spirit and scope of the claimed invention. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

It may be appreciated that the various systems, methods, and apparatus disclosed herein may be embodied in a machine-readable medium and/or a machine accessible medium compatible with a data processing system (e.g., a computer system), and/or may be performed in any order.

The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

1. A system, comprising:
a data acquisition device comprising at least one of a body-worn camera wearable by a code enforcement officer, a vehicle-mounted camera in a vehicle of the code enforcement officer, and a vehicle-deployed drone from the vehicle, each configured to capture video data from a real property along with geospatial coordinates of where the video data is captured in a jurisdictional boundary, and a timestamp of when the video data is captured; and
an evidence management server communicatively coupled with the data acquisition device through a network to:
store the video data captured by the data acquisition device along with the geospatial coordinates and the timestamp,
identify a parcel number in the jurisdictional boundary associated with a parcel captured in the video data based on geospatial coordinates, and
determine whether there has been a violation of at least one of a jurisdictional code based on a modification of at least one of a physical structure and a landscaping in the parcel when the timestamp is compared to the immediately previous timestamp of video footage captured associated with the same parcel number.

2. The system of claim 1,
wherein the data acquisition device comprises the body-worn camera and the vehicle-deployed drone launchable from the vehicle, the vehicle-deployed drone being configured to capture an aerial image and the video data of the real property and to transmit the aerial image and the video data with the geospatial coordinates to the evidence management server optionally in real time,
wherein the evidence management server comprises an artificial intelligence module configured to analyze the aerial image and the video data to detect a violation,
wherein the artificial intelligence module is configured to generate a real-time alert to the code enforcement officer upon detecting the violation,
wherein the body-worn camera and the vehicle-mounted camera are configured to operate in a synchronized mode to capture multiple perspectives of the real property simultaneously and to provide time-aligned frames to the evidence management server, and
wherein the evidence management server comprises an evidence-management interface configured to integrate with a municipal database to automatically cross-reference a detected change with a property record and a permit record associated with the parcel number.

3. The system of claim 1,
wherein the evidence management server further comprises a geolocation module configured to associate captured images and the video data with a geographic location of the real property and to map the detected change to a parcel-boundary polygon corresponding to the parcel number,
wherein the evidence management server comprises a computer-vision module configured to detect at least one of: construction of an unpermitted structure, alteration to an exterior building characteristic, accumulation of debris, non-compliant materials, modification to fencing, driveways, and accessory structures,
wherein the evidence-management interface further comprises an automated report generator configured to prepare an enforcement notice and citation based on an identified violation, the enforcement notice and citation including a time-stamped, geo-referenced image frame captured by at least one of the body-worn camera, the vehicle-deployed drone, and an ordinance citation, and
wherein the evidence management server is configured to detect interior modifications of the real property based on imagery obtained through windows, open structures, and authorized interior inspections, and to associate each detection with a confidence score.

4. A system, comprising:
a body-worn camera wearable by a code enforcement officer configured to capture video data of a real property together with a geospatial coordinate and a timestamp;
a vehicle-deployed drone launchable from a vehicle and configured to capture an aerial image and the aerial video data of the real property together with the geospatial coordinates and the timestamp; and
an evidence management server communicatively coupled with the body-worn camera and the vehicle-deployed drone through a network, the evidence management server configured to:
receive the video data from the body-worn camera and the aerial video data from the vehicle-deployed drone;
time-synchronize the video data and the aerial video data;
identify a parcel number associated with the real property based on the geospatial coordinates;
compare a time-synchronized data with the video data associated with an immediately previous timestamp for the parcel number to detect a modification to at least one of a physical structure and a landscaping;
evaluate a detected modification against digitized municipal code sections and cross-reference the detected modification with the real property and permit records retrieved from a municipal database;
generate a real-time alert responsive to a determination of non-compliance; and
create an enforcement notice comprising geo-referenced imagery captured by at least one of the body-worn camera and the vehicle-deployed drone.

5. The system of claim 4,
wherein the evidence management server comprises a municipal dashboard configured to present active violations, owner information, prior permits, and prior violations for the parcel number, and a priority ranking based on at least one of a severity level, a location risk factor, and a duration since detection,
wherein the evidence management server further comprises a change-detection module configured to perform at least one of semantic segmentation, pixel-wise differencing, and bounding-box generation to isolate a detected portion and to compute at least one measurement selected from width, height, surface area, and footprint boundary, and to overlay the at least one measurement on the image and the video data, and
wherein the vehicle-deployed drone comprises a gimbal-stabilized camera and a telemetry transceiver and is configured to stream live geo-tagged video to the evidence management server.

6. The system of claim 4,
wherein the evidence management server is further configured to command the vehicle-deployed drone to reposition to capture a supplemental aerial view of the detected portion and to fuse the supplemental aerial view with a time-synchronized video from the body-worn camera,
wherein the evidence management server is configured to transmit an automated notification of a violation via at least one of an electronic mail, a short message service, a municipal web portal, and a printed mail, and to schedule a site visit by a municipal staff member responsive to the real-time alerts,
wherein the evidence management server is configured to assemble an evidence package comprising the enforcement notice, before-and-after imagery with overlaid measurements, the geospatial coordinates, an applicable ordinance citation, and chain-of-custody metadata including a cryptographic hash and a tamper-evident audit log,
wherein the evidence management server comprises a machine-learning model trained on historical enforcement outcomes and property imagery and configured to update detection performance by periodic retraining using labeled compliant and non-compliant events, and
wherein the body-worn camera and the vehicle-deployed drone are time-synchronized using a common clock distributed by the evidence management server to enable frame-level correlation and multi-perspective fusion.

7. A computer-implemented method for real-time evidence management, the method comprising:
storing in a jurisdictional code database a jurisdictional regulation in an evidence management server comprising any one or more of a planning code, a building code, a construction code, a public works code, a cleanliness code, an administrative code, a statutory code, a fire code, a police code, an ordinance, a safety code, a housing code, a municipal code,
capturing a video data of a real property using at least one of a body-worn camera worn by a code enforcement officer, a vehicle-mounted camera in a vehicle of the code enforcement officer, and a vehicle-deployed drone launched from the vehicle, the video data including a geospatial coordinate within a jurisdictional boundary and a timestamp of capture;
transmitting the video data, the geospatial coordinates, and a timestamp from a data acquisition device to the evidence management server through a network;
storing the video data together with the geospatial coordinates and the timestamp in the evidence management server;
identifying a parcel number in the jurisdictional boundary that corresponds to the real property captured in the video data based on the geospatial coordinates; and
determining whether a violation of the jurisdictional regulation has occurred by comparing a timestamped video data with the video data from an immediately previous timestamp associated with the same parcel number to detect a modification to at least one of a physical structure and a landscaping of a parcel.

8. The method of claim 7,
wherein the data acquisition device comprises a multispectral imaging module configured to capture at least one of infrared imagery, thermal imagery, and LiDAR point clouds, and
wherein the evidence management server is configured to detect structural changes not visible in RGB imagery, including at least one of roof degradation, heat leakage, and energy code violations.

9. The method of claim 7, further comprising an acoustic sensing module configured to detect non-compliant sound patterns, the non-compliant sound patterns comprising at least one of construction machinery operated outside permitted hours, amplified sound levels exceeding municipal noise ordinances, and unpermitted industrial equipment.

10. The method of claim 7,
wherein the evidence management server is communicatively coupled to at least one environmental sensor configured to detect air quality, dust generation, light emissions, and waste disposal conditions, and to evaluate compliance with an environmental regulation associated with the parcel number, and
wherein the evidence management server is configured to fuse the video data from the data acquisition device with a resident-reported imagery, the resident-reported imagery comprising photographs, video, and sensor recordings submitted through a municipal reporting portal, to corroborate detection of a jurisdictional code violation.

11. The method of claim 7, wherein an artificial intelligence module comprises a natural language processing engine configured to automatically map a detected modification to a specific section of a digitized municipal ordinance and regulatory code.

12. The method of claim 7, wherein the artificial intelligence module comprises a consensus engine configured to execute a plurality of trained detection models and to issue a violation alert only upon multi-model consensus exceeding a confidence threshold.

13. The method of claim 7, wherein the evidence management server is further configured to generate a three-dimensional parcel model by aggregating repeated aerial captures from the vehicle-deployed drone, and to detect volumetric changes to the parcel, including footprint expansion, height increases, and demolition.

14. The method of claim 7, wherein the evidence management server comprises a heat-mapping module configured to visualize detected violations on a jurisdictional map, the heat-mapping module assigning severity levels and risk indicators to prioritize enforcement actions.

15. The method of claim 7, wherein a machine-learning model is further configured to retrain adaptively using enforcement outcomes, including citations upheld and dismissed, thereby improving future detection accuracy.

16. The method of claim 7, wherein the evidence management server overlays detected parcel modifications against zoning maps to identify mismatches between actual property usage and designated zoning classifications.

17. The method of claim 7, wherein an evidence package comprises a multi-tier encrypted copy of sensitive resident-facing imagery, the multi-tier encryption comprising a redacted public copy and an unredacted secured chain-of-custody copy.

18. The method of claim 7, wherein the evidence package further comprises a digitally watermarked image frame, the digitally watermarked image frame embedding the parcel number, ordinance citation, the timestamp, and the geospatial coordinates directly into an evidentiary image.

19. The method of claim 7, wherein the evidence management server is configured to provide an augmented reality overlay to a display associated with the body-worn camera, the overlay visually superimposing detected violations and ordinance references onto a real-world scene in real time.

20. The method of claim 7, wherein the data acquisition device comprises a voice annotation module enabling the code enforcement officer to dictate contextual notes while recording, the notes being transcribed, geotagged, and automatically linked to the corresponding evidentiary record.
